(19) United States
(12) Patent Application Publication
(10) Pub. No.: US 2026/0223885 A1
(43) Pub. Date: Aug. 6, 2026

(54) EDIBLE MIXOTROPHIC-BORNE PROTEINIC FOOD PRODUCTS AND METHODS THEREOF

(71) Applicant: BREVEL LTD., Kiryat Gat (IL)

(72) Inventors: Ido GOLAN, Rehovot (IL); Amir LEVI, Rehovot (IL); Shlomit PELED, Rehovot (IL); Yonathan AZOULAY, Kibbutz Lahav (IL)

(21) Appl. No.: 19/149,687

(22) PCT Filed: Jan. 18, 2024
(86) PCT No.: PCT/IL2024/050078

§ 371 Date: Jul. 21, 2025

(30) Foreign Application Priority Data
Jan. 19, 2023 (IL) 300066

(51) Int. Cl. A23J 1/00; A21D 2/26; A23C 11/06; A23C 20/00; A23G 9/38; A23J 3/20; A23J 3/22; A23L 15/00; A23L 21/25; C12N 1/12

(52) U.S. Cl. CPC A23J 1/009; A21D 2/267; A23C 11/065; A23C 20/00; A23G 9/38; A23J 1/008; A23J 3/20; A23J 3/227; A23L 15/35; A23L 21/27; C12N 1/12

(57) ABSTRACT

The present invention discloses production means, process methods, and edible mixotrophic-borne proteinic product utilizable as an ingredient, beverage or food product, produced in a closed photobioreactor for growing a microorganism culture in an aqueous medium.

### FIELD OF THE INVENTION

The present invention generally pertains to edible mixotrophic-borne proteinic products utilizable as ingredients, beverages or food products. The invention also relates with method of producing the same in a closed photobioreactor for growing a microorganism culture in an aqueous medium.

### BACKGROUND OF THE INVENTION

Civilization faces significant challenges in the years ahead. The population growth predicted over the next few decades a global population of about 9.7 billion by 2050 will cause severe food shortages. Malnutrition is a leading cause of world-wide death, accounting for about 3.5 million deaths per year. Global deforestation will cause the loss of a significant portion of the food currently relied on. The resources currently utilized are unsustainable; two planets worth of resources will be needed to support the expected population by 2050. Accordingly, there is a significant need to identify sustainable alternatives to the world's current food resources. For example, food sources that can provide improved functionality, higher nutritional value, minimal waste stream, reduced water usages, and reduced carbon dioxide emissions are needed. Microalgae are a rich source of protein, essential fatty acids, vitamins, and minerals. After lipid removal, the residual biomass contains even higher concentrations of protein and other nutrients. Microalgae are good sources of long chain polyunsaturated fatty acids (“PUFA”) and have been used to enrich diets with omega-3 PUFAs.

A type of microalgae named Euglena (as disclosed e.g., in WO2022189976A1 which discloses) belongs to a group of single-celled microscopic algae, that is often used as a candidate species for laboratory studies and technological applications. Mixotrophic growth, in which carbon sources are assimilated together with CO₂ does not occur in all species. Chlorella (disclosed below) is a genus of single-celled green algae belonging to the division Chlorophyta. Chlorella I spherical in shape, about 2 to 10 μm in diameter, and is without flagella. It contains the green photosynthetic pigments chlorophyll-a and-b in its chloroplast. Chlorella multiples rapidly, requiring only carbon dioxide, water, sunlight, and a small amount of minerals to reproduce. The terms “food”, “food product” and “food composition” mean a product or composition that is intended for ingestion by an animal (human or other animals, such as pets, aquafarming- and farm-animals) and provides at least one nutrient to the animal. The term refers in a non-limiting manner to cold-served, hoot-served, served in ambient conditions, fluidic, at least partially fluidic, semi rigid or rigid (solid) products, being homogeneously or heterogeneously made, potentially comprising aggregates, colloids, oil phase, water miscible phase, O/W, W/O, W/O/W or O/W/O suspensions, or any combination thereof.

Chlorella is a potential food source since it is high in protein and other essential nutrients. For example, when dried, chlorella contains about 45% protein, 20% fat, 20% carbohydrate, 5% fiber, and 10% minerals and vitamins (e.g., vitamin B12, vitamin C, iron, magnesium, zinc, copper, potassium, and/or calcium, etc.). Due to this, chlorella has been labeled as a “superfood” and has garnished significant attention from the vegan community. Further, chlorella has been explored as a potential source of food and energy because its photosynthetic efficiency can, in theory, reach 8%, which exceeds that of other highly efficient crops, such as sugar cane, see e.g., US20220204571A1 which discloses a method for using peptides for flavorings, comprising steps of obtaining a microalgae; extracting chlorella protein from the microalgae; modifying a factor associated with the chlorella protein to change an amino acid combination of the chlorella protein; and identifying a peptide flavoring associated with the modified amino acid combination. Currently, there are an estimated 300,000 to 1 million species of microalgae in existence. Microalgae has recently attracted considerable interest due to their extensive applications in the renewable energy field, the biopharmaceutical field, and the nutraceutical field. Specifically, microalgae may be a sustainable and economical source of biofuels, bioactive medicinal products, and food ingredients. Moreover, microalgae also have applications in wastewater treatment and atmospheric CO₂ mitigation. Thus, microalgae produce a wide range of bioproducts, including polysaccharides, lipids, pigments, proteins, vitamins, bioactive compounds, and antioxidants; see US20220204917A1 which discloses production of functional protein using microalgae in mixotrophic and/or heterotrophic cultivation.

It is a long felt need to have edible mixotrophic-borne proteinic products utilizable as ingredients, beverages or food products, especially those produced in closed photobioreactor for growing a microorganism culture in an aqueous medium.

### SUMMARY OF THE INVENTION

It is an object of the present invention to disclose an edible mixotrophic-borne product characterized in that it comprises a composition of mixotrophic borne edible proteins from mixotrophic microorganisms. The microorganisms are cultured under uninterrupted continuous mixotrophic conditions in a photobioreactor with intensity of light, wavelengths at the range of 300 and 700 nm, between 100 to 7,000 lux/liter; flux of photons to the surface is between 10 to 2500 micro-Einstein (μE, m⁻²s⁻¹), growth medium along the at least last three to six, especially five days of pre-harvesting comprises glucose as the organic carbon source at a concentration of 0.1 to 40 grams per liter. The composition is selected from Group A of proteins originated from and produced by the mixotrophic microorganisms, the intensity of the mixotrophic borne proteins of Group A is at least two times greater than heterotrophic borne proteins of Group A if they were produced by the same microorganism grown in heterotrophic conditions. The composition is further selected from Group B of proteins originated from and produced by the mixotrophic microorganisms, the intensity of mixotrophic borne proteins of Group B is at least two times greater than autotrophic borne proteins of Group B if they were produced by the same microorganism grown in photoautotrophic conditions.

It is another object of the invention to disclose a mixotrophic-borne product as defined above, and selected form a group consisting of food and beverage product(s) comprising meat/fish analog, eggs analog, milk analog, including cheese, yogurt, bakery and pasta product, including breads and cakes, honey analog and any combination, derivative and mixture thereof.

It is another object of the invention to disclose an edible mixotrophic-borne proteinic product for the food industry product characterized as defined in any of the above.

It is another object of the invention to disclose the edibles as defined in any of the above, comprises Ribulose-1,5-bisphosphate carboxylase/oxygenase (RuBisCo) where the percentage of RuBisCo in the composition is at a range of 5 to 100 percentages of the total dry weight.

It is another object of the invention to disclose the edibles as defined in any of the above, wherein the protein is a member of a group consisting of a whole mixotrophic microorganism, portions of the mixotrophic microorganism, water miscible extracts from the mixotrophic microorganism or portions thereof, water immiscible extracts from the mixotrophic microorganism or portions thereof, and any combination, derivatives and mixtures thereof.

It is another object of the invention to disclose the edibles as defined in any of the above, wherein the edibles are a member of a group consisting of ingredient, beverage or food product, the method comprising steps of cultivating a microorganism under mixotrophic conditions in closed photobioreactor for growing a microorganism culture in an aqueous medium.

It is another object of the invention to disclose a method of obtaining an edible mixotrophic-borne product, wherein the method comprising steps of culturing microorganisms under uninterrupted continuous mixotrophic conditions in a photobioreactor with intensity of light, wavelengths at the range of 300 and 700 nm, between 100 to 7,000 lux/liter; flux of photons to the surface is between 10 to 2500 micro-Einstein (μE, m-2s-1); providing growth medium along the at least last three to six, especially five days of pre-harvesting with glucose as the organic carbon source at a concentration of 0.1 to 40 grams per liter. The hereto cultured composition of mixotrophic borne edible proteins from mixotrophic microorganisms is characterized by that for Group A of proteins originated from—and produced by—the mixotrophic microorganisms, the intensity of the mixotrophic borne proteins of Group A is at least two times greater than heterotrophic borne proteins of Group A if they were produced by the same microorganism grown in heterotrophic conditions. The hereto cultured composition of mixotrophic borne edible proteins from mixotrophic microorganisms_is further characterized by that for Group B of proteins originated from and produced by the mixotrophic microorganisms, the intensity of mixotrophic borne proteins of Group B is at least two times greater than autotrophic borne proteins of Group B if they were produced by the same microorganism grown in photoautotrophic conditions.

It is another object of the invention to disclose a method of obtaining an edible mixotrophic-borne proteinic product for the food industry product. The method comprising the steps defined above.

It is another object of the invention to disclose a method as defined in any of the above, characterized by that it comprises steps of (a) growing microalgae mixotrophic strains in culturing plates under predetermined controlled mixotrophic growth conditions, to a desirable growth rate; (b) transferring the microalgae culture into larger culturing vessel for further growing under controlled predetermined mixotrophic growth conditions, to a desirable growth rate; (c) transferring the micro microalgae culture into larger culturing bioreactor for further growing under predetermined controlled mixotrophic growth conditions, to a desirable growth rate; and (d) transferring the microalgae culture into industrial-scale photobioreactor designed for commercial production purposes, for further growing under predetermined controlled mixotrophic growth conditions and production of proteinic products.

It is another object of the invention to disclose a method as defined in any of the above, wherein at least one of the n following conditions is/are held true: (a) growth medium contains an organic carbon source at a concentration of about 0.1 to about 40 grams per liter; (b) growth medium also contains organic or inorganic nitrogen and phosphate; (c) high flux of photons is maintained throughout the entire process, from the plate stage to the industrial reactor, with light intensity between about 100 to about 7,000 lux/liter; (d) flux of photons to the surface is between about 10 to about 500 μE, m⁻²s⁻¹; (e) lighting wavelengths provided are in the photosynthetic range (PAR) and can include specific wavelengths in the range between about 300 and about 700 nm; (f) Temperature is maintained between about 10 degrees and about 40 degrees Celsius; and (g) pH of the culture medium is maintained within the range of about 4 to about 10.

It is another object of the invention to disclose a method_as defined in any of the above, wherein n is an integer selected from a group consisting of 1, 2, 3, 4, 5, 6, and 7.

It is another object of the invention to disclose a method as defined in any of the above, wherein the edible mixotrophic-borne proteinic product is a member of a group consisting of the whole mixotrophic-fermented microorganism, portions of the mixotrophic-fermented microorganism, water miscible extracts from the mixotrophic-fermented microorganism or portions thereof, water immiscible extracts from the mixotrophic-fermented microorganism or portions thereof, and any combination, derivatives and mixtures thereof.

It is another object of the invention to disclose a method as defined in any of the above, wherein the edible mixotrophic-borne proteinic product is a member of a group consisting of meat analog, food product comprising meat analog, eggs analog, food product comprising eggs analog, milk analog, food product comprising milk analog, including cheese, yogurt, bakery and pasta product, including breads and cakes, honey analog and food product and confitures comprising honey analog, and any combination, derivative and mixture thereof.

It is another object of the invention to disclose a method as defined in any of the above, wherein the edible mixotrophic-borne proteinic product is a member of a group consisting of ingredient, beverage or food product, the method comprising steps of fermenting a microorganism under mixotrophic conditions in closed photobioreactor for growing a microorganism culture in an aqueous medium.

### BRIEF DESCRIPTION OF THE FIGURES

The accompanying figures, which are incorporated in and constitute a part of this specification, illustrate aspects of the invention and, together with a general description of the invention given above, and the detailed description given below, serve to explain the invention, wherein

FIG. 1a, and FIG. 1b [for Set 1 (k) and Set 2 (y), respectively] disclose signal intensity for the detected proteins; y-axis is signal intensity, x-axis is peptide number, according to one set of embodiments of the invention;

FIG. 2 illustrates an NMD plot showing similarity in protein composition and expression between the different treatments according to other sets of embodiments of the invention; and

FIGS. 3a-3b for Group A and B, respectively, depict the absolute expression of two protein groups: Group A (mixotrophs to heterotrophs intensity ratio >5); whereas Group B (mixotrophs to autotrophs intensity ratio >5), respectively. Mixotrophs are blue bars. Heterotrophs are orange bars, and Autotrophs are grey bars (Values are average of two replicates), according to yet other sets of embodiments of the invention

### DETAILED DESCRIPTION OF THE PREFERRED EMBODIMENTS

Some species that enhance their growth under mixotrophy (interchangeably hereinafter

“Mixotrophic species” and “Mixotrophic microorganisms”) are selected from a group consisting of aforementioned Chlorella sp., C. vulgaris, C. protothecoides; Asterarcys sp., Nannochloropsis salina, N. gaditana, N. oleoabundans, Haematococcus sp., Botryococcus braunii, D. salina, P. purpureum, P. tricornutum and species and combinations thereof; see e.g., tables 1 and 5, in Castillo, Tania, et al. “Mixotrophic cultivation of microalgae: An alternative to produce high-value metabolites.” Biochemical Engineering Journal 176 (2021): 108183, incorporated herein as a reference. Additionally, or alternatively, other non-limiting examples of mixotrophic microalgae and cyanobacteria for mixotrophic growth using acetic acid or acetate as an organic carbon source may comprise organisms of the genera: Chlorella, Anacystis, Synechococcus, Synechocystis, Neospongiococcum, Chlorococcum, Phaeodactylum, Spirulina, Micractinium, Haematococcus, Nannochloropsis, Brachiomonas, and species and combinations thereof. Additionally, or alternatively, other non-limiting examples of mixotrophic microalgae and cyanobacteria comprise organisms of the genera: Agmenellum, Amphora, Anabaena, Anacystis, Apistonema, Arthrospira {Spirulina), Botryococcus, Brachiomonas, Chlamydomonas, Chlorella, Chloroccum, Cruciplacolithus, Cylindrotheca, Coenochloris, Cyanophora, Cyclotella, Dunaliella, Emiliania, Euglena, Extubocellulus, Fragilaria, Galdieria, Goniotrichium, Haematococcus, Halochlorella, Isochyrsis, Leptocylindrus, Micr actinium, Melosira, Monodus, Nostoc, Nannochloris, Nannochloropsis, Navicula, Neospongiococcum, Nitzschia, Odontella, Ochromonas, Ochrosphaera, Pavlova, Picochlorum, Phaeodactylum, Pleurochyrsis, Porphyridium, Poteriochromonas, Prymnesium, Rhodomonas, Scenedesmus, Skeletonema, Spumella, Stauroneis, Stichococcus, Auxenochlorella, Cheatoceros, Neochloris, Ocromonas, Porphyridium, Synechococcus, Synechocystis, Tetraselmis, Thraustochytrids, Thalassiosira, and species and combinations thereof. Additionally or alternatively, in some embodiments the microorganisms or compositions may comprises a portion of microorganisms comprise one or more of the following carboxydotrophic microorganisms: Acinetobacter sp.; Alcaligenes carboxydus or other Alcaligenes sp., Arthrobacter sp., Azomonas sp., Azotobacter sp., Bacillus schlegelii or other Bacillus sp Hydrogenophaga pseudoflava or other Hydrogenophaga sp Pseudomonas carboxydohydrogena, Pseudomonas carboxydovorans, Pseudomonas compransoris, Pseudomonas gazotropha, Pseudomonas thermocarboxydovorans, or other Pseudomonas sp Rhizobium japonicum or other Rhizobium sp and Streptomyces G26, Streptomyces thermoautotrophicus, or other Streptomyces sp as defined in WO2021151025A1. In certain embodiments, a carboxydotrophic microorganism is used. In certain embodiments, a carboxydotrophic microorganism that is capable of chemolithoautotrophy is used. In certain embodiments, a carboxydotrophic microorganism that is able to utilize H as an electron donor in respiration and/or biosynthesis is used. Additionally, heterotrophic microalgae and cyanobacteria, such as but not limited to Schizochytriuml Aurantiochytrium, may be used in some embodiments of the described continuous system, see WO2017218996A1, incorporated herein as a reference, which discloses methods of culturing microalgae in a continuous auxostat system useful in mixotrophic culture conditions.

As used herein, the terms ‘mixotroph’, ‘mixotrophy’ or ‘mixotrophic’ mixotrophic-borne′ refer to organisms, processes, compositions, by-products, raw-materials, and products which can use- or be provided by-more than one source of energy and/or organic compounds. Most often, this refers to organisms, processes and products provided or yielded by microorganisms which can use a mixture of light and chemical inputs to acquire or produce energy and/or organic compounds. Mixotrophic organisms exist on a spectrum between full obligate chemoheterotrophy and full obligate photoautotrophy. Using such a mixture of sources may be obligate, where an organism must use the mixture of sources to survive, or facultative, where the organism uses one source preferentially and the other under particular circumstances, for example using chemical sources of energy where light is limiting. Therefore, a ‘mixotrophic microorganism’ is both a phototroph and a chemotroph, and may be a photoautotroph, a chemoautotroph, a photoheterotroph, or a chemoheterotroph.

Mixotrophic species, under mixotrophy conditions, are known to yield (‘Mixotrophic-borne’-) pigments and other terpenoids, polyunsaturated fatty acids (ω-3 and ω-6) such as arachidonic (ARA; C20:4 ω-6), eicosapentaenoic (EPA; 20:5 ω-3) and docosahexaenoic (DHA; 22:6 ω-3), in which their biosynthesis takes place in the chloroplast, cytosol and endoplasmic reticulum. Other bioactive compounds of biotechnological importance, produced by Mixotrophic species include vitamins, polysaccharides, peptides, polyphenols and phytosterols. The protein content of microalgal biomass can reach approximately 30-50%, some are obtained by enzymatic digestion of microalgal biomass (e.g., Chlorella, Nannochloropsis and Tetraselmis) by proteolytic enzymes. Some of the peptides have shown potential therapeutic activities, such as antioxidant, anti-inflammatory, anticancer, antimicrobial, and antihypertensive activities.

A few patent applications presents means and method for providing effective bioengineering for mixotrophic cultivation, se for example WO2018011809A1, WO2017012933A1 which discloses biomass of Thraustochytrids (Aurantiochytrium mangrovei CCAP 4062/2; CCAP 4062/3; CCAP 4062/4; CCAP 4062/5; CCAP 4062/6; CCAP 4062/1; and Schizochytrium sp. 4087/3; CCAP 4087/1; CCAP 4087/4; CCAP 4087/5) comprising proteins and fatty acids, characterized in that it comprises, by weight relative to the weight of the dry matter, at least 35% of proteins and at least 20% of fatty matter, and in that the biomass is biomass without added proteins or fatty acids.

The terms “microbiological culture”, “microbial culture”, or “microorganism culture” interchangeable refer to a method or system for multiplying Mixotrophic microorganisms, such as microalgae and cyanobacteria, through reproduction in a predetermined culture medium, including under controlled laboratory conditions. Microbiological cultures, microbial cultures, and microorganism cultures are used to multiply the organism, to determine the type of organism, or the abundance of the organism in the sample being tested. In liquid culture medium, the term microbiological, microbial, or microorganism culture generally refers to the entire liquid medium and the microorganisms in the liquid medium regardless of the vessel in which the culture resides. A liquid medium is often referred to as “media”, “culture medium”, or “culture media”. Nutrients in microorganism culture media may comprise nitrogen, phosphorus, micronutrients, trace metals, and vitamins. Many recipes for culture media can be found in the public domain, such as BG-11 media and f/2 media. The act of culturing is generally referred to as “culturing microorganisms” when emphasis is on plural microorganisms. The act of culturing is generally referred to as “culturing a microorganism” when importance is placed on a species or genus of microorganism. Microorganism culture is used synonymously with culture of microorganisms.

Organic carbon sources suitable for growing microalgae and cyanobacteria mixotrophically or heterotrophically, may comprise: acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolysate, proline, propionic acid, ribose, saccharose, partial or complete hydrolysates of starch, sucrose, tartaric, TCA-cycle organic acids, thin stillage, urea, industrial waste solutions, yeast extract, and combinations thereof, see WO2017218996A1. The organic carbon source may comprise any single source, combination of sources, and dilutions of single sources or combinations of sources.

In certain embodiments, many of the defined by alternation from WO2021138482A1, a nutrient media for culture growth and production is used, comprising an aqueous solution containing suitable minerals, salts, vitamins, cofactors, buffers, and other components needed for microbial growth, known to those skilled in the art, see Bailey and Ollis, Biochemical Engineering Fundamentals, 2nded; pp 383-384 and 620-622; McGraw-Hill: New York (1986). In certain embodiments, the chemicals used for maintenance and growth of mixotrophic microbial cultures as known in the art are included in the nutrient media. In certain embodiments, these chemicals may include but are not limited to one or more of the following: nitrogen sources such as ammonia, ammonium (e.g., ammonium chloride, ammonium sulfate, nitrate (e.g., potassium nitrate), urea or an organic nitrogen source; phosphate (e.g., disodium phosphate, potassium phosphate, phosphoric acid, potassium dithiophosphate, potassium orthophosphate, dipotassium phosphate); sulfate; yeast extract; chelated iron; potassium (e.g., potassium phosphate, potassium nitrate, potassium iodide, potassium bromide); and other inorganic salts, minerals, and trace nutrients (e.g., sodium chloride, magnesium sulfate or magnesium chloride, calcium chloride or calcium carbonate, manganese sulfate or manganese chloride, ferric chloride, ferrous sulfate or ferrous chloride, sodium bicarbonate or sodium carbonate, zinc sulfate or zinc chloride, ammonium molybdate or sodium molybdate, cuprous sulfate or copper chloride, cobalt chloride, aluminum chloride, lithium chloride, boric acid, nickel chloride, tin chloride, barium chloride, copper selenate or sodium selenite, sodium metavanadate, chromium salts. In certain embodiments, the mineral salts medium (MSM) formulated by Schlegel et al may be used, see e.g., “Thermophilic bacteria”, Jakob Kristjansson, Chapter 5, Section III, CRC Press, (1992).

The term “cultured meat” refers to any and all interchangeable terms, including healthy meat, vegetarian meat, slaughter-free meat, in vitro meat, vat-grown meat, lab-grown meat, cell-based meat, clean meat, meat analog, cultivated meat, synthetic meat, and artificial meat. As used herein, and in a non-limiting manner, the term also refers to a food product that is not derived from an animal, or that contains a substantial amount of non-animal protein source, but has structure, texture, aesthetic qualities, and/or other properties comparable or similar to those of animal meat, including livestock (e.g., beef, pork), game (e.g., venison), poultry (e.g., chicken, turkey, duck), and/or fish or seafood substitutes/analogs. The term also refers to uncooked, cooking, and cooked meat-like food product. The term also refers to an eidible material which comprises muscle (protein-) and fat replicas.

In most cases, cultivated meat comprises vegetable protein, such protein from chickpea, faba bean, lentils, lupine, mung bean, pea or soy, protein materials from oil seed, such as hemp, rapeseed/canola or sunflower, protein materials from cereals, such as rice, wheat or triticale, further potato protein, and protein materials from plant leaves, such as alfalfa leaves, spinach leaves, sugar beet leaves or water lentil leaves, and algae protein and mixtures thereof. A few studies, see e.g., WO2022171647A1, EP4095626A1 EP4039796A1 used microbial proteins, which are also termed single cell proteins (SCP) include fungal proteins, also termed mycoproteins, such as proteins from Fusarium venenatum, proteins from yeast such as proteins from Saccharomyces species, proteins from algae, such as proteins from spirulina or Chlorella vulgaris, species, and bacterial proteins, such as proteins from lactobacilli species.

There is no current publication of cultured meat made of mixotrophic species, excluding a few prior-art, such as US20210392920A1 and WO2021151025A1 which generally and nonspecifically mentions mixotrophic growth of microorganisms does not provides embodiments and other means for such a production.

In accordance with an embodiment, the mixotrophic-borne protein product is combined with other edible ingredients to form a food product, including an artificial meat product which mimics one or more physical characteristics and/or functional properties of meat, such as texture, flavor, aroma, and/or appearance. Such other ingredients may be selected from apple cider, apple cider vinegar, baking powder, baking soda, beans, beef, beet juice, beet powder, black pepper, brown sugar, butter, canola oil, caramel, carrot fiber, carrots, cashews, cheese, chicken, chocolate, citrus, citrus extract, coconut oil, condensed milk, dairy, egg, egg substitute, fish, flour, garbanzo bean, garlic powder, honey, liquid smoke, maple syrup, margarine, monosodium glutamate, mustard powder, oil, olive oil, onion powder, paprika, pork, potato, potato starch, rice flour, salt, sodium benzoate, soy (protein and/or oil), soy sauce, spices, spirulina, sugar, sunflower oil, tomato juice, tomato powder, tomato sauce, tomatoes, turmeric, vanilla, vinegar, vitamins and minerals, walnuts, water, wheat, wheat flour, wheat gluten, xanthan gum, yeast, and/or yeast extract.

The term “about” as used herein when referring to a measurable value such as an amount, a temporal duration, and the like, is meant to encompass variations of =5%, +1%, or =0.1% from the specified value, as such variations are appropriate to perform the disclosed methods or in connection with a disclosed composition.

The term “biomass” refers to a material produced by growth and/or propagation of cells. Biomass may contain cells and/or intracellular contents as well as extracellular material, including, but not limited to, compounds secreted by a cell.

The term “bioreactor” or “fermenter” refers to a closed or partially closed vessel in which cells are grown and maintained. The cells may be, but are not necessarily, held in liquid suspension. In some embodiments, rather than being held in liquid suspension, cells may alternatively be grown and/or maintained in contact with, on, or within another non-liquid substrate including but not limited to a solid growth support material. As used herein, “contain(s) substantially no (or substantially free of)” means containing substantially none of the substance in question, but it does not mean containing absolutely none of the substance, and for example, glucose may be present at less than 10 mg/L as the minimum limit of determination, amino acids (such as L-glutamine) may be present at less than 0.1 mg/L as the minimum limit of determination, or vitamins may be present at less than 1 μg/L as the minimum limit of determination.

The term “culturing” (or cultured) refers to growing a population of mixotrophic microorganisms, under suitable conditions for growth, in a liquid or solid medium. The term is also used for cultivating, fermenting, bio-reacting, producing, and growing.

The term “derived from” encompasses the terms “originated from,” “obtained from,” “obtainable from,” “isolated from,” and “created from,” and generally indicates that one specified material finds its origin in another specified material or has features that can be described with reference to another specified material.

The phrase “substantially free” or “essentially free” as to any given component means that such component is only present, if at all, in an amount that is a functionally insignificant amount, i.e., it does not significantly negatively impact the intended performance or function of any process or product. Typically, substantially free means less than about 1%, including less than about 0.5%, including less than about 0.1%, and also including zero percent, by weight of such component. The terms “substantially free” or “essentially free” shall me less than 1% of a component.

The term “Peptide” refers to a mixotrophic-borne peptide (a mixotrophic-borne polypeptide) consisting of two or more amino acids linked in a chain, the carboxyl group of each acid being joined to the amino group of the next by a bond of the type R—OC—NH—R′, for example, about 2 amino acids to about 50 amino acids, or 21 amino acids to about 50 amino acids. As used herein, “polypeptide” refers to a mixotrophic-borne composition comprised of amino acids and recognized as a protein by those of skill in the art. The conventional one-letter or three-letter code for amino acid residues is used herein. The terms “polypeptide” and “protein” are used interchangeably herein to refer to mixotrophic-borne polymers of amino acids of any length. The polymer may be linear or branched, it may comprise modified amino acids, and it may be interrupted by non-amino acids. The terms also encompass mixotrophic-borne amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, unnatural amino acids, etc.), as well as other modifications known in the art.

In accordance with an embodiment, the mixotrophic-borne protein product comprises at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, or at least about 85% of the artificial meat product on a dry basis, e.g., by weight on a dry basis.

The term “yield” refers to amount of a mixotrophic-borne product produced from a feed material relative to the total amount of the substance that would be produced if all of the feed substance were converted to product. For example, yield of the product may be expressed as % of the product produced relative to a theoretical yield if 100% of the feed substance were converted to the product.

High mixotrophic-borne protein food products—In some embodiments, high mixotrophic-borne protein food compositions are provided as well as methods of making the same. A “protein product” (e.g., one or more of single cell protein, cell lysate, protein concentrate, protein isolate, protein extract, protein hydrolysate, free amino acids, peptides, oligopeptides, or combinations thereof), derived from one or more mixotrophic microorganisms described herein, may be processed or incorporated into a high-protein mixotrophic-borne edible food composition for human and/or animal consumption. A food composition (food product, this term includes beverages and any other drinkable food product) may be, for example, a food item, and/or a food ingredient, and/or a nutritional product, and/or an animal feed, and/or a pet food product.

In some embodiments, the food composition may contain any of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% mixotrophic-borne microbial protein product, as described herein, by weight, e.g., by weight on a dry weight basis. In certain embodiments, the mixotrophic-borne protein products as described herein are utilized in the production of a vegetarian or vegan food product. In certain embodiments, they are utilized in the production of an organic food product and/or pesticide-free and/or herbicide-free and/or fungicide-free and/or antibiotic-free and/or non-genetically modified (non-GMO) food product. In certain embodiments, they are utilized in a locally produced food product. In certain embodiments, they are utilized in a probiotic food product or in a prebiotic food product (e.g., prebiotic nutritional product).

Processes are provided for making a high protein mixotrophic-borne food product, which includes one or more mixotrophic-borne protein product from one or more mixotrophic microorganism as described herein. In certain embodiments, the food mixotrophic-borne product has no animal protein or fats. In certain embodiments, the mixotrophic-borne protein product(s) are incorporated into food mixotrophic-borne products including, but not limited to, dairy products, dairy replacement products, meat products (including livestock, game, poultry, fish, or seafood products), meat replacement and/or imitation meat products (including imitation livestock, game, poultry, fish, or seafood products), bakery products, confections, health and protein bars, protein powders, sports and/or energy drinks, and/or protein shakes and/or smoothies. In certain embodiments, mixotrophic-borne protein products are textured for incorporation into meat products and/or imitation meat products. In certain embodiments, the high mixotrophic-borne protein ingredient can be used as a meat extender in beef patties.

In certain embodiments, a high mixotrophic-borne protein food product as described herein does not include animal fats. In certain embodiments, it has a relatively high ratio of polyunsaturated fats to saturated fats. In certain embodiments, it has a high-quality mixotrophic-borne protein content, roughly comparable to milk protein. In certain non-limiting embodiments, its amino acid content is substantially similar, very close, or identical to that recommended by the United Nations Food and Agriculture Organization as ideal. In certain embodiments, food products made using the protein products of the present invention represent healthy and/or low-calorie foods. In certain embodiments, the protein product has a bland flavor and/or a light cream color and/or easy dispersibility and/or a relatively high-water absorption and/or relatively high fat adsorption. In certain embodiments, the protein product can be formed into fibers and/or thermally extruded and/or coagulated into a gel. In certain such nonlimiting embodiments, gel coagulation occurs at pH falling in a range of about 3 to about 6 upon heating. In certain embodiments, one or more properties of the protein product makes it well suited for incorporation into food products, including but not limited to dairy products, dairy replacement products, meat products, meat replacement and/or imitation meat products, bakery products, confections, health and protein bars, protein powders, sports and/or energy drinks, and/or protein shakes and/or smoothies. In certain embodiments, the protein product is textured for incorporation into meat products and/or imitation meat products. In certain embodiments, the protein product can be used as a meat extender, for example, as a meat extender in beef patties. In certain non-limiting embodiments, roughly 30 parts of the mixotrophic-borne protein product can be combined with 70 parts of meat, e.g., ground beef, and in other embodiments, roughly 10 parts of protein product per 90 parts of meat, e.g., ground beef. In certain non-limiting embodiments, the protein product is combined with beef and/or other meat products in a ratio that conforms to the requirements set forth by the USDA and/or in accordance with regulations governing the National School Lunch Program (Type A School Lunch). In certain non-limiting embodiments, the protein product is included in a formulation having a combined protein equivalence ratio (PER) of around 2.6. In certain non-limiting embodiments, the water absorption and/or fat binding properties of the mixotrophic-borne protein product aids in reducing shrinkage (fat and water loss) on cooking and/or enhances the moisture and texture of the cooked patty or other meat or food item.

In certain embodiments, mixotrophic-borne protein product produced as described herein is included in a recipe and/or formulation along with one or more of the following ingredients: water; tomatoes; tomato juice; tomato sauce; beans; spices including but not limited to chili spice; seasoning; animal protein; beef, poultry (e.g., chicken, turkey, duck, goose); pork; fish; seafood; soy; wheat; flour; yeast; yeast extract; spirulina; margarine; butter; dairy; cheese; sugar; brown sugar; honey; egg; salt; vanilla; chocolate; baking soda; baking powder; condensed milk; and/or caramel. In certain embodiments the combined ingredients are subjected to one or more of hydrating; blending; mixing; beating; sifting; sprinkling; heating; cooking; frying; deep frying; baking; simmering; browning; boiling. In certain embodiments the ingredients are fried at around 350° F. and/or are baked or cooked at around 375° F. to 450° F. In certain embodiments, the mixotrophic-borne protein product is used as a meat extender or meat substitute in one or more of the following: patties; chili con carne; pizza toppings; ground beef; chicken nuggets; fish sticks. In certain embodiments as a meat extender and/or substitute, the protein product replaces about 50% or more than about 50% (e.g., any of at least about 50%, at least about 55%, at least about 60%, at least about 65%, at least about 70%, at least about 75%, or at least about 80%) of the meat ingredient in the food item, in other embodiments they replace less than 50% (e.g., any of less than about 45%, less than about 40%, less than about 35%, less than about 30%, less than about 25%, less than about 20%, less than about 15%, or less than about 10%).

In certain embodiments, the mixotrophic-borne protein product ingredient imparts improved nutrition, water absorption, fat binding properties, texture, and/or eating qualities to a food product, such as a cereal based product. In certain embodiments, the protein product ingredient is used to fortify or is otherwise incorporated into a cereal based product. In certain the embodiments, the cereal-based product is a breakfast cereal, cookie, cake, pie, brownie, muffin, or bread. In certain embodiments, the mixotrophic-borne protein product is used as a replacement for milk proteins (e.g., sodium caseinate) and/or as a vitamin and/or mineral supplement in milk or dairy products. In certain such embodiments, the mixotrophic-borne protein product ingredient is used in one or more of non-fat dried milk, powdered milk, or dairy type drinks, such as, but not limited to, instant breakfast mixes, or imitation dairy type drinks including but not limited to soy milk, rice milk, and almond milk. In certain embodiments, the mixotrophic-borne protein product ingredient is used in nutritionally fortified (e.g., protein, vitamin, and/or mineral fortified) candies, deserts, or treats.

In some embodiments, mixotrophic-borne protein product (e.g., one or more of single cell protein, cell lysate, protein concentrate, protein isolate, protein extract, protein hydrolysate, free amino acids, peptides, oligopeptides, or combinations thereof), derived from one or more mixotrophic microorganisms described herein, is processed to produce a food product or ingredient thereof, in a process that includes heating the mixotrophic-borne protein product, optionally in combination with other ingredients such as, for example, plant-derived protein, under shearing agitation, followed by extrusion to produce a product of desired texture (e.g., chewy, crunchy, crispy, resists dispersion in water, etc.). In exemplary, non-limiting embodiments, an aqueous paste of mixotrophic-borne protein product (for example, containing about 20% (w/w) to about 50% (w/w), about 20% (w/w) to about 40% (w/w), about 30% (w/w) to about 50% (w/w), about 20% (w/w) to about 35% (w/w) or about 35% (w/w) to about 50% (w/w) water), optionally in combination with plant-based materials such as vegetable protein (such as, for example, soybean meal, sesame meal, cottonseed meal, corn meal, wheat meal, and/or peanut meal), is heated to a temperature of about 150° F. to about 400° F., or about 225° F. to about 275° F., for about 10 seconds to about 300 seconds, applying a shearing force, optionally simultaneously with the heating, for example, with a shear rate of about 10 rpm to about 60 rpm and torque of about 200 to about 2,000 meter-grams, and extruding the heated and sheared protein product through a die to provide a shaped extrudate. Optionally, the extrudate is exposed to an oxygen-containing gas stream. In an embodiment, the oxygen-containing gas stream is an air stream (e.g., a dry air stream), for example, at a temperature of about 80° ° F. to about 212° F. for about 0.5 minutes to about 10 minutes.

In some embodiments, mixotrophic-borne protein product (e.g., one or more of single cell protein, cell lysate, protein concentrate, protein isolate, protein extract, protein hydrolysate, free amino acids, peptides, oligopeptides, or combinations thereof), derived from one or more mixotrophic microorganisms described herein, is processed to produce a food product or ingredient thereof, in a process that includes combining the mixotrophic-borne protein product with one or more additional protein source (such as, but not limited to, pea, rice, glutinous rice, wheat, gluten, soy, hemp, canola, insects, algae, and/or buckwheat), heating the mixture (e.g., at about 150° F. to about 400° F., and subjecting the mixture to a shearing force with an extruder to create a textured product with desired textural and/or functional characteristics (e.g., chewy, crunchy, crispy, resists dispersion in water, etc.).

In some embodiments, free amino acids are included, either as part of the mixotrophic-borne protein product or supplemental to the protein product, to impart a desired flavor. In one non-limiting embodiment, glutamic acid is included, thereby imparting a umami flavor to the food product.

In some embodiments, for example, in a mixotrophic-borne meat substitute or artificial meat mixotrophic-borne product, a hydrogel, lipogel, and/or emulsion is included in the composition, for example, as an agent release system (e.g., for release of a coloring agent, a flavor agent, a fatty acid, a leavening agent, a gelling agent (e.g., bicarbonate (e.g., potassium bicarbonate), calcium hydroxide, and/or alginate (e.g., sodium or potassium alginate)), wherein the agent(s) may be released during cooking of the food product to simulate animal meat).

In some embodiments, a food mixotrophic-borne product includes one or more plant protein source such as, but not limited to, pea, rice, glutinous rice, wheat, gluten, soy, hemp, canola, insects, algae, and/or buckwheat, in combination with a protein product produced by microorganisms as described herein (e.g., one or more of single cell protein, cell lysate, protein concentrate, protein isolate, protein extract, protein hydrolysate, free amino acids, peptides, oligopeptides, or combinations thereof), wherein the protein product imparts a flavor to the food composition, such as, for example, a meat-like flavor (including a livestock, game, poultry, or seafood meat-like flavor).

In some embodiments, a food mixotrophic-borne product, for example, a meat substitute or artificial meat product, includes a heme compound, such as a heme-containing polypeptide. In one embodiment, the food product includes heme (e.g., heme-containing polypeptide) from the microorganism from which the protein product is derived. In certain such embodiments the said microorganism is a Cupriavidus microorganism, such as Cupriavidus necator.

In some embodiments, a mixotrophic-borne meat substitute or mixotrophic-borne artificial or imitation meat product (e.g., a livestock (e.g., beef, pork), game, poultry, fish, or seafood analog product) includes a protein product produced by microorganisms as described herein (e.g., one or more of single cell protein, cell lysate, protein concentrate, protein isolate, protein extract, protein hydrolysate, free amino acids, peptides, oligopeptides, or combinations thereof). In some embodiments, the meat analogue product is a vegan product that does not contain any ingredients from animal sources. In some embodiments, an enhanced meat product which contains animal protein (e.g., a beef, poultry, pork, fish, seafood, or egg product, in which a portion of the product is a protein product ingredient produced by microorganisms as described herein (e.g., one or more of single cell protein, cell lysate, protein concentrate, protein isolate, protein extract, protein hydrolysate, free amino acids, peptides, oligopeptides, or combinations thereof)), is provided. For example, the mixotrophic-borne protein product may be included as an extender in an enhanced meat product or in a meat analogue product, e.g., the protein product replaces any of at least about 10%, at least about 20%, at least about 30%, at least about 40%, at least about 50%, at least about 60%, or at least about 70% of the meat ingredient or an artificial or imitation meat ingredient (for example, a plant-based artificial or imitation meat analogue ingredient) to produce the enhanced meat product or meat analogue/imitation meat product, respectively. In some embodiments, the microorganisms are mixture of mixotrophic species and CO₂-grown or air-grown microorganisms, e.g., oxyhydrogen microorganisms.

In some embodiments, at least a portion, all, or substantially all of the mixotrophic-borne protein product in a food product described herein, including but not limited to, a mixotrophic-borne meat substitute or mixotrophic-borne artificial meat product, includes protein product (e.g., one or more of single cell protein, cell lysate, protein concentrate, protein isolate, protein extract, protein hydrolysate, free amino acids, peptides, oligopeptides, or combinations thereof) derived from a Cupriavidus microorganism, such as, but not limited to, Cupriavidus necator, e.g., DSM 531 or DSM 541.

In some embodiments, at least a portion, all, or substantially all of the mixotrophic-borne protein product in a food product described herein, including but not limited to, a mixotrophic-borne meat substitute or mixotrophic-borne artificial meat product, includes mixotrophic-borne protein product with or without others, (e.g., one or more of single cell protein, cell lysate, protein concentrate, protein isolate, protein extract, protein hydrolysate, free amino acids, peptides, oligopeptides, or combinations thereof) derived from a lactic acid bacterium, such as, but not limited to a Lactococcus, Lactobacillus, Enterococcus, Streptococcus, or Pediococcus bacterium. In some embodiments, the lactic acid bacterium is a Generally Recognized as Safe (GRAS) bacterium.

In some embodiments, at least a portion or all of the mixotrophic-borne protein product in a food product described herein, including but not limited to, a mixotrophic-borne meat substitute or mixotrophic-borne artificial meat product, includes protein product (e.g., one or more of single cell protein, cell lysate, protein concentrate, protein isolate, protein extract, protein hydrolysate, free amino acids, peptides, oligopeptides, or combinations thereof) derived from a Fusarium, Rhizopus, or Aspergillus fungal microorganism, such as but not limited to, Fusarium venenatum, Rhizopus oligosporus, Rhizopus oryzae, Aspergillus oryzae, or Apergillus sojae. In some embodiments, the fungal microorganism is a GRAS microorganism.

mixotrophic-borne Protein products A mixotrophic-borne protein product (e.g., one or more of mixotrophic-borne single cell protein, mixotrophic-borne cell lysate, mixotrophic-borne protein concentrate, mixotrophic-borne protein isolate, mixotrophic-borne protein extract, mixotrophic-borne protein hydrolysate, mixotrophic-borne free amino acids, mixotrophic-borne peptides, mixotrophic-borne oligopeptides, or combinations thereof), is derived from and/or includes biomass and/or mixotrophic-borne protein isolate, mixotrophic-borne protein extract, mixotrophic-borne protein hydrolysate, mixotrophic-borne free amino acids, mixotrophic-borne peptides, and/or mixotrophic-borne oligopeptides derived from one or more mixotrophic microorganisms described herein.

In some embodiments, the mixotrophic-borne protein product includes free amino acids. In certain embodiments, mixotrophic-borne amino acids are produced by mixotrophic and other means known or provided by applying changes from the known in the art, see e.g., WO2014/145194, which is incorporated herein by reference in its entirety.

In some embodiments, the mixotrophic-borne protein product exhibits water and/or oil absorption at a level that is suitable for incorporation into a food composition as described herein, such as, but not limited to a mixotrophic-borne artificial meat or mixotrophic-borne meat substitute composition. For example, the water holding capacity of the protein product may be about 1 to about 10, e.g., about 2 to about 4, times by weight.

In one embodiment, the mixotrophic-borne protein product includes heme (e.g., heme-containing polypeptide), which is produced by the mixotrophic microorganism from which the protein product is derived, mixotrophic-borne and non-mixotrophic-borne such as a Cupriavidus microorganism, e.g., Cupriavidus necator.

In some embodiments, at least a portion, all, or substantially all of the mixotrophic-borne protein in a mixotrophic-borne protein product described herein, including but not limited to, mixotrophic-borne single cell protein, mixotrophic-borne cell lysate, mixotrophic-borne protein concentrate, mixotrophic-borne protein isolate, mixotrophic-borne protein extract, mixotrophic-borne protein hydrolysate, mixotrophic-borne free amino acids, mixotrophic-borne peptides, and/or mixotrophic-borne oligopeptides, is derived from mixotrophic-borne and non-mixotrophic-borne species such as Cupriavidus microorganism, e.g., Cupriavidus necator, e.g., DSM 531 or DSM 541.

In some embodiments, at least a portion, all, or substantially all of the mixotrophic-borne protein in a mixotrophic-borne protein product described herein, including but not limited to, mixotrophic-borne single cell protein, mixotrophic-borne cell lysate, mixotrophic-borne protein concentrate, mixotrophic-borne protein isolate, mixotrophic-borne protein extract, mixotrophic-borne protein hydrolysate, mixotrophic-borne free amino acids, mixotrophic-borne peptides, and/or mixotrophic-borne oligopeptides, is derived from mixotrophic-borne organism, optionally in combination with non-mixotrophic-borne lactic acid bacterium, such as, but not limited mixotrophic microorganisms together with other non-mixotrophic species, such as Lactococcus, Lactobacillus, Enterococcus, Streptococcus, or Pediococcus bacterium. In some embodiments, the lactic acid bacterium is a GRAS bacterium.

In some embodiments, at least a portion, all, or substantially all of the mixotrophic-borne protein product in a p mixotrophic-borne protein product described herein, including but not limited to mixotrophic microorganisms together with other non-mixotrophic species, such as single cell protein, cell lysate, protein concentrate, protein isolate, protein extract, protein hydrolysate, free amino acids, peptides, and/or oligopeptides, is derived from a Fusarium, a Rhizopus, or an Aspergillus fungal microorganism, such as but not limited to, Fusarium venenatum, Rhizopus oligosporus, Rhizopus oryzae, Aspergillus oryzae, or Aspergillus sojae. In some embodiments, the fungal microorganism is a GRAS microorganism.

mixotrophic-borne Protein concentrates In certain embodiments, methods are employed that extract mixotrophic-borne non-protein fractions (e.g. lipids, nucleic acids, polysaccharides) without solubilizing the major protein fractions. The insoluble protein fractions that are recovered are protein concentrates. In certain embodiments, a protein concentrate is produced from protein-containing biomass produced by one or more microorganisms as described herein.

In certain embodiments, a mixotrophic-borne protein concentrate is produced via a solvent extraction process. In certain such embodiments, the solvent extraction process comprises an alcohol extraction or wash, such as, for example, an aqueous alcohol wash. In certain embodiments an acid treatment is utilized to produce a protein concentrate. In certain embodiments a protein concentrate is produced via a heat denaturation process.

In certain embodiments, one or more of solvent extraction, acid treatment, and/or heat denaturation steps are deployed, and may be used sequentially or in parallel for production of a mixotrophic-borne protein concentrate. In certain embodiments, a solvent extraction step is followed by a heat denaturation step in the production of a mixotrophic-borne protein concentrate. In certain embodiments, a heat denaturation step is followed by a solvent extraction step in the production of a mixotrophic-borne protein concentrate. In certain embodiment a heat denaturation step and an acid treatment are combined in the production of a mixotrophic-borne protein concentrate. In certain such embodiments, an insoluble material resulting from heat plus acid treatment is subjected to a solvent extraction step. In certain embodiments, a mixotrophic-borne protein concentrate produced via one or more of solvent extraction, acid treatment, and/or heat denaturation step(s) is subjected to a water wash.

In certain embodiments, a mixotrophic-borne protein concentrate, as described herein contains at least a portion, or most, of the oil and/or water-soluble non-protein constituents that were present in the starting biomass removed by the mixotrophic-borne protein concentrate process.

In certain embodiments, a mixotrophic-borne protein concentrate, as described herein contains at least about 60%, at least about 70%, at least about 80%, or at least about 90% protein by weight, on a moisture free basis. The crude mixotrophic-borne protein content may be defined as the total nitrogen weight percentage (% N) of a material multiplied by a Jones factor equal to 6.25, i.e., crude protein=6.25*% N. In certain embodiments, a mixotrophic-borne protein concentrate, as described herein contains a crude mixotrophic-borne protein content of at least about 60%, at least about 70%, at least about 80%, or at least about 90% mixotrophic-borne protein by weight, on a moisture free basis. The determination of the total amino acid content of a proteinaceous material is well established in the science of biochemical analysis (e.g., using AOAC method 994.12). In certain embodiments, a mixotrophic-borne protein concentrate, as described herein contains a total amino acid content of at least about 60%, at least about 70%, at least about 80%, or at least about 90% by weight of the concentrate, on a moisture free basis. In certain embodiments, a mixotrophic-borne protein concentrate produced according to the present invention contains a higher mixotrophic-borne protein content and/or higher crude mixotrophic-borne protein content and/or higher total amino acid content, than a soy protein concentrate.

In certain embodiments, a mixotrophic-borne protein concentrate, as described herein contains a carbohydrate content of less than about 20%, less than about 10%, less than about 5%, or less than about 1% by weight. In certain embodiments, a mixotrophic-borne protein as described herein contains an ash content of less than about 10%, less than about 8%, less than about 5%, less than about 4%, less than about 3%, less than about 2%, or less than about 1% by weight. In certain embodiments, a mixotrophic-borne protein concentrate, as described herein contains a lipid content of less than about 10%, less than about 8%, less than about 5%, less than about 4%, less than about 3%, less than about 2%, or less than about 1% by weight.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents. This invention includes all modifications and equivalents of the subject matter recited in the claims and/or aspects appended hereto as permitted by applicable law.

### Example 1

### Mixotrophic Vs (Autotrophic and Heterotrophic) Cultivation

Autotrophic and heterotrophic cultivation are the two main strategies in the commercial production of microalgae. Heterotrophic growth relies on organic compounds (mainly glucose) as soul energy and carbon sources, while autotrophic cultures use light and CO₂. Mixotrophic cultivation describes a growth protocol that combines the two and uses both light and sugar as source of energy and carbon.

The Proteome is the entire set of proteins present in an organism at a given time. Proteomics analysis allows us to compare the biological state of the microalgae. Here we present proteomic analysis for the three treatments. 2 sets of samples (biological replicates) each.

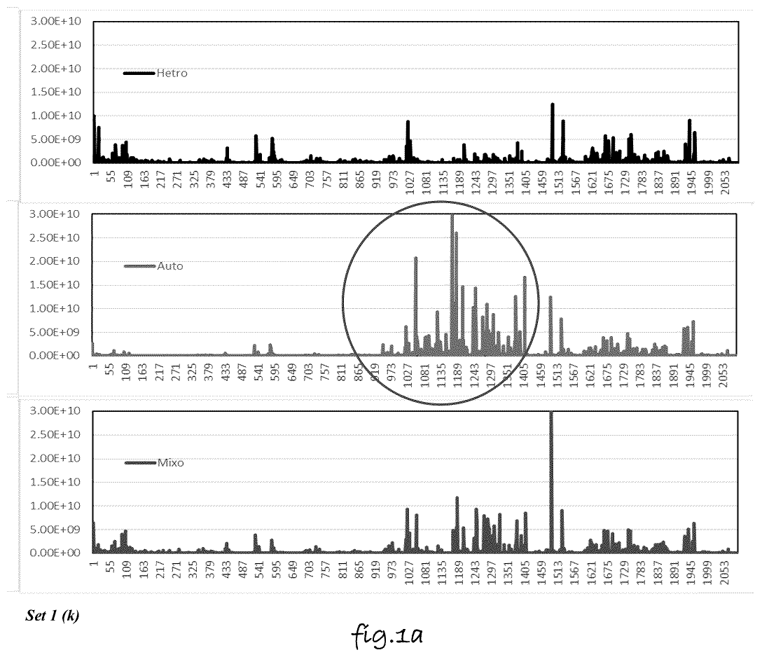

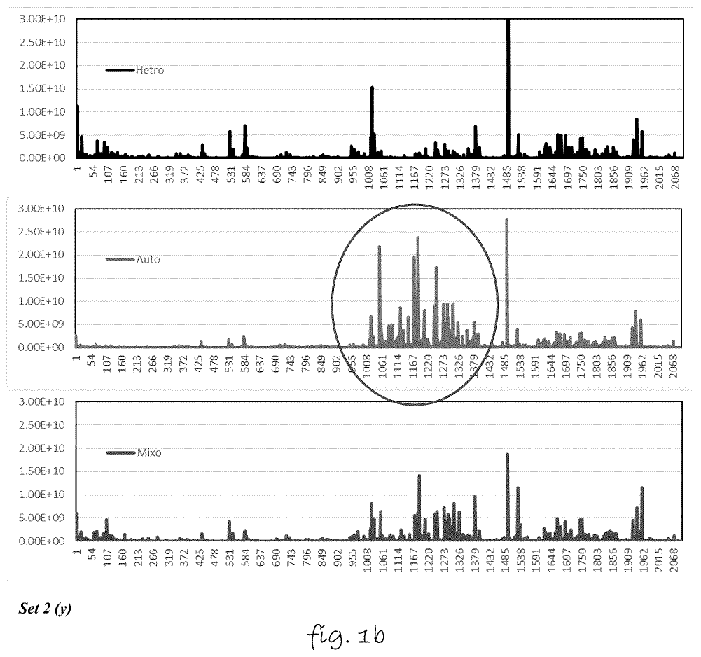

The term intensity commonly refers to either the height or area of the chromatographic or spectral peak; and in the context of mass spectra, it refers to the number of ions that hit the detector per unit of time (seconds). The intensities used in obtaining the quantification ratios and performing the analyses can be computed in a number of different ways. The most suitable intensity measure typically depends upon the type of data acquired. A simple measure is the maximum intensity value of the identified peak. Alternatively, the intensity can be the peak area (or volume for three-dimensional data). It is to be understood that the term “intensity,” as used herein, refers to intensity measures computed in any desired manner; see e.g., U.S. Pat. No. 6,835,927B2. Reference is now made to both Set 1 (k) FIG. 1a, and Set 2 (y) FIG. 1b. Signal intensity for the detected proteins is presented in the following histograms (y-axis is signal intensity, x-axis is peptide number), indicating variability between sample sets. Furthermore, the heterotrophic samples (black histograms) are characterized by high number of proteins with relatively low signal. The autotrophic samples (green histograms) are characterized by a group of proteins with high signal which are circled in the histogram, whereas the mixotrophic samples (red histograms) present an in-between pattern.

FIG. 1a, and Set 2 (y) FIG. 1b. Signal intensity for the detected proteins is presented in the following histograms (y-axis is signal intensity, x-axis is peptide number), indicating variability between sample sets

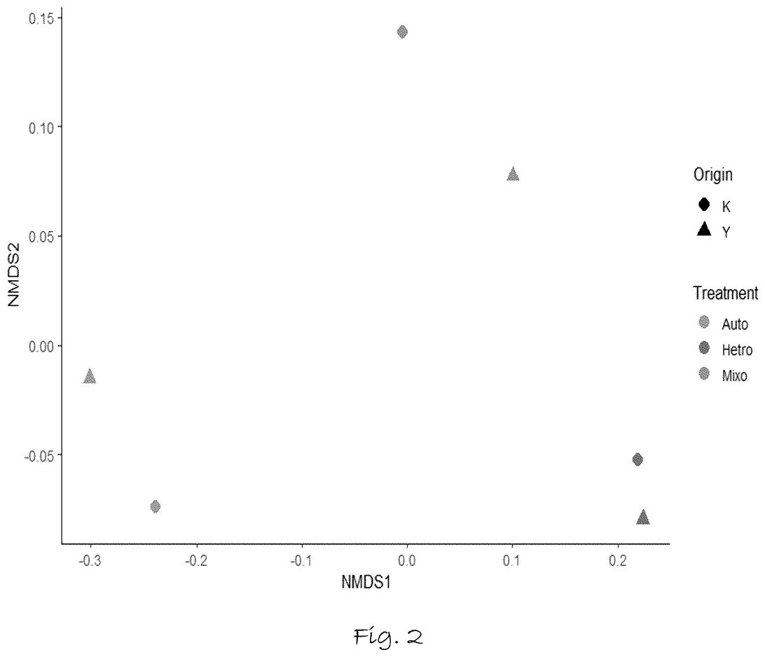

(Non-metric Multidimensional Scaling (NMD) graph: Reference is now made to FIG. 2, illustrating an NMD plot to graphicly present the similarity in protein composition and expression between the different treatments. The more similar they are the distance between them reduce. In this plot, each treatment cluster separately from the other, demonstrating the shift in protein expression due to growth protocol. Moreover, the biological replicates cluster close together, which supports the initial claim.

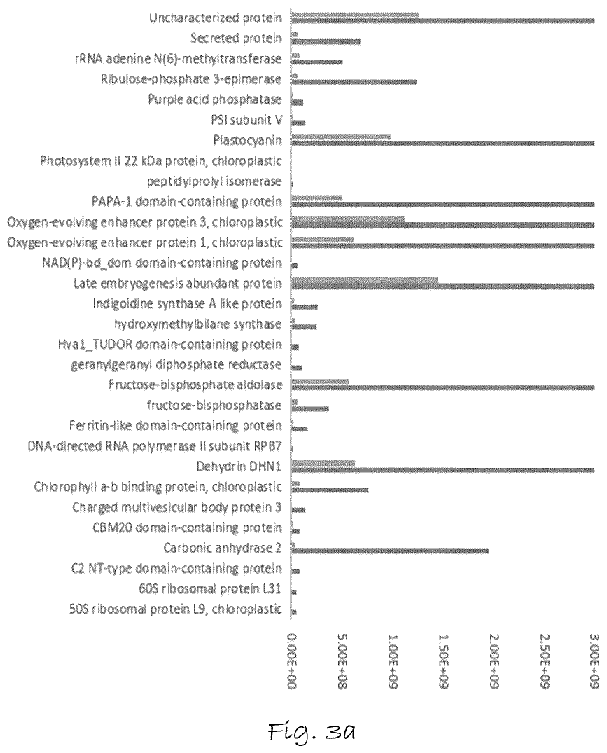

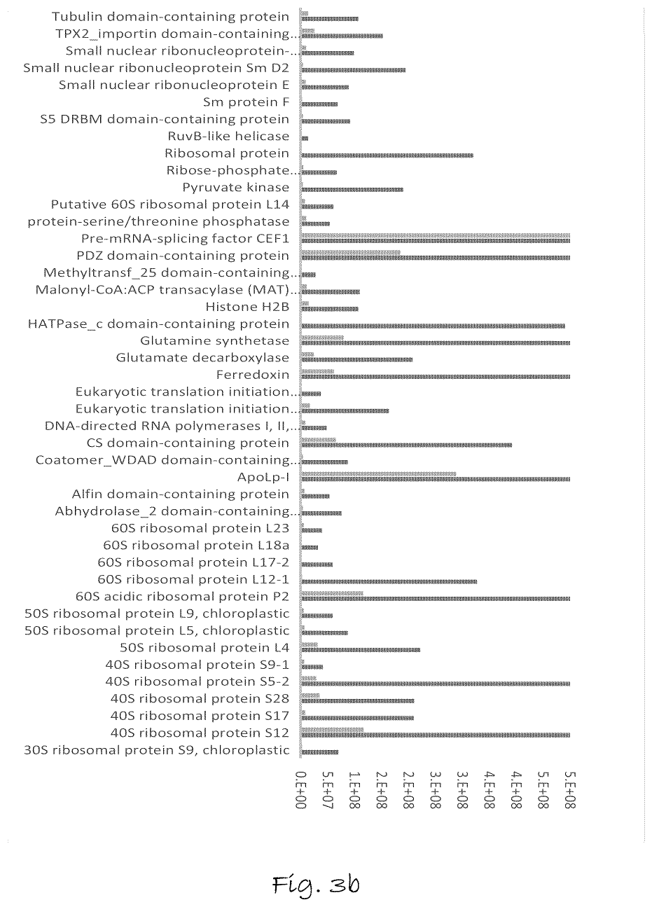

Over expression comparison: Reference is now made to FIGS. 3a-3b. The bar graphs present the absolute expression of two protein groups: Group A (mixo/hetero intensity ratio >5); whereas Group B (mixo/auto intensity ratio >5), respectively. Mixotrophs are blue bars. Heterotrophs are orange bars, and Autotrophs are grey bars (Values are average of two replicates).

Group A contain 30 over expressed proteins, among them chloroplast proteins associated with the photosynthetic machinery. This includes Chl a/b binding proteins, several Photosystem II core proteins, Oxygen-evolving enhancer and plastocyanin. This is potentially due to the degeneration of the photosystem under heterotrophic conditions.

Group B on the other hand contains some 44 overexpressed proteins among those several subunits of the ribosome and some metabolic enzymes such as pyruvate kinase and Glutamine synthetase. Together could imply faster cell division due to better energetic conditions.

FIGS. 3a-3b are depicted by high expression of this combination (cell division related proteins and Photosystem related proteins) can be the unique protein finger print of mixotrophic growth.

### Example 2

### Biological Conditions of Mixotrophic Cultivation

Strains of algae used for cultivation are mixotrophic strains. Growth medium contains glucose as the organic carbon source at a concentration of about 0.1 to about 40 grams per liter. The growth medium also contains organic or inorganic nitrogen and phosphate. A high flux of photons is maintained throughout the entire process, from the plate stage to the industrial reactor, with light intensity between about 100 to about 7,000 lux/liter. The flux of photons to the surface is between about 10 to about 2,500 micro-Einstein (μE m⁻²s⁻¹). The lighting wavelengths provided are in the photosynthetic range (PAR) and can include specific wavelengths in the range between about 300 and about 700 nm. Temperature is maintained between about 20 degrees and about 30 degrees Celsius. pH of the culture medium is maintained within the range of about 6.4 to about 8.

### Example 3

### Scale-Up Steps in Mixotrophic Cultivation

It is the scope of an embodiment of the invention wherein the scaled up process comprises, inter alia, steps as follows: Utilizing mixotrophic strains of algae; Starting with mixotrophic growth; Inoculating the microalgae in a mixotrophic growth condition; transferring them from the plate stage to an Erlenmeyer flask or similar container; Transferring to a larger reactor: Once the microalgae have reached a suitable growth stage in the Erlenmeyer flask, transferring the culture to a larger-scale reactor; Scaling up to an industrial reactor; Transferring the microalgae culture from the previous reactor to an industrial-scale reactor designed for commercial production purposes.

It is the scope of yet another embodiment of the invention wherein the process comprises steps of maintaining a high ratio of surface area to lighting intensity is crucial in our mixotrophic cultivation process. This strategic approach ensures that the algae receive sufficient and uniform light exposure throughout the culture, which plays a vital role in inducing the desired components and achieving the unique protein profile. By distributing the light sources effectively over the culture area and optimizing the surface area to the culture, we enhance light penetration and create an ideal environment for the algae's growth and protein expression. This high surface area to lighting intensity ratio ensures that the algae receive the necessary photons for photosynthesis and protein. As the cell density increases, we maintaining an optimal lighting environment. By increasing the lighting intensity in response to the higher cell density, we ensure that the light threshold required for the induction of the photosynthetic system is consistently met. This adjustment of lighting allows us to sustain efficient photosynthetic activity even in denser cultures, promoting a robust expression of the photosynthetic components in the algae.

LIST 1 Group A of proteins intensity where mixotrophic growth
to heterotroph growth ratio is at least five time greater

Secreted protein
rRNA adenine N(6)-methyltransferase
Ribulose-phosphate 3-epimerase
Purple acid phosphatase
PSI subunit V
Plastocyanin
Photosystem II 22 kDa protein, chloroplastic
peptidylprolyl isomerase
PAPA-1 domain-containing protein
Oxygen-evolving enhancer protein 3, chloroplastic
Oxygen-evolving enhancer protein 1, chloroplastic
NAD(P)-bd_dom domain-containing protein
Late embryogenesis abundant protein
Indigoidine synthase A like protein
hydroxymethylbilane synthase
Hva1_TUDOR domain-containing protein
geranylgeranyl diphosphate reducatase
Fructose-bisphosphate aldolase
fructose-bisphosphatase
Ferritin-like domain-containing protein
DNA-directed RNA polymerase II subunit RPB7
Dehydrin DHN1
Chlorophyll a-b binding protein, chloroplastic
Charged multivesicular body protein 3
CBM20 domain-containing protein
Carbonic anhydrase 2
C2 NT-type domain-containing protein [symbol]
ribosomal protein L31 [symbol]
ribosomal protein L9, chloroplastic [symbol]
indicates data missing or illegible when filed

LIST 2 Group B of proteins intensity where mixotrophic growth
to autotroph growth ratio is at least two to five time greater.

Tubulin domain-containing protein
TPX2_importin domain-containing [symbol]
Small nuclear ribonucleoprotein [symbol]
Small nuclear ribonucleoprotein Sm D2
Small nuclear ribonucleoprotein E
Sm protein F
S5 DRBM domain-containing protein
RuvB-like helicase
Ribosomal protein
Ribose-phosphate [symbol]
Pyruvate kinase
Putative [symbol] ribosomal protein L14
protein-serine/threonine phosphatase
Pre-mRNA-splicing factor CEF1
PDZ domain-containing protein
Methyltransf_25 domain-containing [symbol]
Malonyl-CoA:ACP transacylase (MAT) [symbol]
Histone H2B
HATPase_c domain-containing protein
Glutamine synthetase
Glutamate decarboxylase
Ferredoxin
Eukaryotic translation initiation [symbol]
Eukaryotic translation initiation [symbol]
DNA-directed RNA polymerases [symbol]
C [symbol] domain-containing protein
Coatomer_WDAD domain-containing [symbol]
ApoLp-1
Alfin domain-containing protein
Abyhydrolase_2 domain-containing [symbol]
6 [symbol] ribosomal protein L23
6 [symbol] ribosomal protein L18a
6 [symbol] ribosomal protein L17-2
6 [symbol] ribosomal protein L12-1
6 [symbol] acidic ribosomal protein P2
5 [symbol] ribosomal protein L9, chloroplastic
5 [symbol] ribosomal protein L5, chloroplastic
5 [symbol] ribosomal protein L4
4 [symbol] ribosomal protein [symbol] 9-1
4 [symbol] ribosomal protein [symbol] 5-2
4 [symbol] ribosomal protein [symbol] 28
4 [symbol] ribosomal protein [symbol] 17
4 [symbol] ribosomal protein [symbol] 12 [symbol]
ribosomal protein [symbol] 9, chloroplastic [symbol]
indicates data missing or illegible when filed

### Example 4

### Mixotrophic-Borne (Vegetarian-) Meat and Meat Analogue Products

In some embodiments, an artificial mixotrophic-borne meat product includes at least about 10%, at least about 15%, at least about 20%, or at least about 25% by weight of microbial protein product as described herein, optionally bound together by one or more binding agents, to produce a food product that has one or more similar textural and/or functional characteristics in comparison to animal meat. In some embodiments, the mixotrophic-borne artificial meat product resembles animal meat, for example, ground animal meat (e.g., ground beef, ground pork, ground turkey). In some embodiments, the mixotrophic-borne artificial meat product is principally or entirely composed of ingredients derived from non-animal sources. In alternative embodiments, the artificial meat product is composed of ingredients partially derived from animal sources but supplemented with ingredients derived from non-animal sources. In some embodiments, the mixotrophic-borne artificial meat product further includes one or more agent release systems and/or other ingredients. In various embodiments, mixotrophic-borne artificial meat products herein may be sliced, cut, ground, shredded, grated, or otherwise processed, or left unprocessed. Examples of sliced forms include but are not limited to dried meats, cured meats, and sliced lunch or deli meats. In some embodiments, the mixotrophic-borne artificial meat food products provided herein are shredded and then bound together, chunked and formed, ground and formed, or chopped and formed, for example, to produce a product similar in appearance and/or texture to animal jerky.

In some embodiments, the mixotrophic-borne artificial meat products are vegan. In some embodiments, the mixotrophic-borne artificial meat products comprise no GMO ingredients. In some embodiments, the mixotrophic-borne artificial meat products comprise no ingredients derived from nuts. In some embodiments, the mixotrophic-borne artificial meat products comprise less than about 0.6% or less than about 0.5% by weight of sodium. In some embodiments, the mixotrophic-borne meat-like food products comprise no gluten or substantially no gluten. In some embodiments, the mixotrophic-borne meat-like food products comprise no soy or substantially no soy.

In some embodiments, the mixotrophic-borne artificial meat food products provided herein comprise about 5% to about 30% by weight of lipid, e.g., about 5% to about 10%, about 10% to about 15%, about 15% to about 20%, about 20% to about 30%, about 5% to about 15%, about 10% to about 20%, about 20% to about 30%, about 5% to about 15%, about 15% to about 30%, about 5% to about 25%, or about 10% to about 30 by weight of lipid. In some embodiments, the artificial meat products comprise about 0.5% to about 10% by weight of total carbohydrate, e.g., about 0.5% to about 1%, about 1% to about 5%, about 5% to about 10%, about 2% to about 8%, or about 3% to about 6% by weight of total carbohydrate. In some embodiments, the mixotrophic-borne artificial meat products comprise about 0.5% to about 5% by weight of edible fiber, e.g., about 0.5% to about 1%, about 1% to about 5%, about 5% to about 10%, about 2% to about 8%, or about 3% to about 6% by weight of edible fiber.

The mixotrophic-borne artificial meat products provided herein may comprise a moisture content (MC) of at least about 30%, at least about 40%, at least about 50%, at least about 60%, at least about 70%, at least about 80%, or at least about 90% by weight. In some embodiments, the mixotrophic-borne artificial meat products comprise a similar MC as animal meat (e.g., livestock, game, poultry, fish, or seafood meat).

In some embodiments, the mixotrophic-borne artificial meat products comprise one or more coloring agents. In some embodiments, the mixotrophic-borne artificial meat products comprise one or more color enhancers. In some embodiments, the mixotrophic-borne meat-like food products comprise mixtures of two or more coloring agents, color stabilizers, and/or color enhancers. Non-limiting examples of such mixtures include beet extract and annatto, beet extract and turmeric, beet extract and saffron, beet extract and purple carrot, beet extract and grape seed extract, beet extract and tomato extract, beet extract and lycopene, beet extract and beta carotene, beet extract and anthocyanin, beet extract and anthocyanin and annatto, beet extract and annatto and lycopene, beet extract and ascorbic acid, anthocyanin and annatto, beet extract and annatto and ascorbic acid, beet extract and annatto and beta carotene, beet extract and turmeric and ascorbic acid, and anthocyanin and lycopene and annatto. In some such embodiments, the coloring agents, color stabilizers, and/or color enhancers are mixotrophic-borne. In other embodiments, those are present at equal weight ratios. In other such embodiments, the coloring agents, color stabilizers, and/or color enhancers are present at unequal weight ratios (e.g., 55:45, 60:40, 65:35, 2:1, 70:30, 75:25, 80:20, 5:1, 85:15, 90:10, 20:1, 95:5, or 99:1). In some embodiments, the artificial meat products comprise browning agents, such as, but not limited to, pentose (e.g., ribose, arabinose, xylose), hexose (e.g., glucose, fructose, mannose, galactose), dextrins, and commercial browning agents (e.g., red arrow dextrose, wood-derived agents).

In some embodiments, a mixotrophic-borne artificial meat product herein includes one or more plant protein source such as, but not limited to, pea, rice, glutinous rice, wheat, gluten, soy, hemp, canola, insects, algae, and/or buckwheat, in combination with a protein product produced by mixotrophic microorganisms as described herein, wherein the protein product imparts a meat-like flavor to the composition.

In some embodiments, a mixotrophic-borne artificial meat product herein includes a heme compound, such as a heme-containing polypeptide. For example, the heme compound (e.g., heme-containing polypeptide) may be from the microorganism from which the protein product is derived. In certain such embodiments the heme compound is derived from mixotrophic-borne and from non-mixotrophic-borne species, such as a Cupriavidus microorganism, for example, Cupriavidus necator. In certain such embodiments the heme compound is a hemoglobin or flavohemoglobin.

Different meat samples were selected for a comprehensive comparison of mechanical properties between natural, processed, and cultured meat: (1) commercial processed Frankfurt-style sausages (sausage), (2) processed turkey breast cold cut (turkey), (3) non-processed raw breast chicken, and (4) Frankfurt-style sausage made of cultured meat.

### Sloppy Joe Analog Meat Dish

200 grams of mixotrophic-borne Protein, thawed
1/2 chopped hot pepper
1/2 chopped onion
1 chopped garlic clove
1/4 cup ketchup
1 tablespoon of mustard
1/2 cup of water
1 teaspoon chili powder
Salt and pepper to taste
2 hamburger buns

### Shepherd's Pie Analog Meat Dish

a casserole with a layer of cooked meat and vegetables, topped with mashed potatoes, and baked in the oven until the mashed potatoes are well browned.
175 gr of mixotrophic-borne protein
1 large onion
1 cup uncooked rice
1 grated carrot
1 grated zucchini
4-5 generous spoons of tomato sauce (if not then at least add some tomato paste)
1/2 cup bread crumbs
1/2 cup of flour+water mixotrophic-borne batter (with a thick texture, like a pancake)
2 chopped garlic cloves
A handful of chopped parsley
Salt and pepper

### Sausages Analog Meat Dish

250 grams of mixotrophic-borne protein
3 chopped garlic cloves
1/4 cup chopped purple onion or a relatively small onion
1/4 teaspoon cumin
1/4 teaspoon ground cardamom
1/4 ground nutmeg
1/2 teaspoon of black mustard seeds
1/2 teaspoon ground coriander
3/4 teaspoon black pepper
1 teaspoon of salt
1/2 teaspoon cane sugar
3 teaspoons smoked paprika
3 tablespoons olive oil or canola oil
3 tablespoons of soy sauce
5 tablespoons of almond powder
5-10 tablespoons of water
1 cup chickpea flour
1 teaspoon cornflour

### Example 5

### Mixotrophic-Borne (Vegetarian-) Tuna Fish

2×15-ounce mixotrophic-borne protein
1/3 cup vegan mixotrophic-borne mayo
1 tablespoon soy sauce
1 tablespoon mustard
1 tablespoon nori sheet blended into flakes
1/2 medium red onion chopped
1 stalk celery chopped
1 handful parsley chopped
1/2 lemon the juice
1 tablespoon capers or pickles-chopped
2 pinches salt
2 twists black pepper

### Vegan Mixotrophic-Borne Mayo:

200 gr mixotrophic-borne protein
3 tbsp vegan mixotrophic-borne mayo
1/2 red onion
1 celery stick
1 tbsp capers
1/2 lemon
1 tsp wholegrain mustard
1/2 tsp garlic powder
Salt and black pepper

### Example 6

### Mixotrophic-Borne (Vegetarian-) Egg Analog and Products Thereof

A liquid mixotrophic-borne egg analog is an egg substitute with properties resembling that of a conventional egg product. In this description, the “dry weight” refers to the weight of the composition before the introduction of added water. Some amount of water may be naturally present in ingredients before the introduction of added water.

While the composition is generally referred to as a “liquid egg mixotrophic-borne analog”, in some embodiments the composition may be produced and provided as a dry substance, e.g., a powder. A powdered egg mixotrophic-borne analog-equivalently, powdered egg substitute—can be hydrated with a suitable dispersant such as water and/or oils.

Some embodiments may be referred to as a plant-based liquid egg mixotrophic-borne analog. In this description, plant-based is taken to mean that the ingredients in that composition are mostly or entirely made of plant-based foods and not derived from animals. This may mean that ingredients in the composition have non-living sources (e.g., some salts) or living sources that are not animals (e.g., plants, fungi, algae, etc.).

An exemplary liquid egg mixotrophic-borne analog comprises at least a dispersant, a mixotrophic-borne protein, high acyl gellan gum, low acyl gellan gum and an emulsifier. Various additional ingredients may also be included such as additional dispersants, additional proteins, additional hydrocolloids, buffering agents, preservatives, texturizing agents including cation salts, fortifiers, additional emulsifiers, colorants and flavor enhancers. Individual ingredients may fit more than one category. For example, calcium carbonate may be included and serve as a texturizing agent (cation salt) and as a fortifier.

Mixotrophic-borne (vegetarian-) egg analog -
A first receipt (adopted from WO2023/279189)

| Liquid Egg Analog | Dry Weight | | Total Weight | | |
|---|---|---|---|---|---|
| Ingredient | (%) | | (%) | | Description |
| Water | | | 76.2 | (74-78) | Dispersant |
| A first mixotrophic- | 42.68 | (40-44) | 10.15 | (10-2) | Functional protein |
| borne protein | | | | | |
| Sunflower Oil* | 29.02 | (27-31) | 6.9 | (6.5-7) | Dispersant |
| A first and/or a second | 8.41 | (7-9) | 2.00 | (1.75-2.25) | Functional protein |
| mixotrophic-borne | | | | | |
| protein | | | | | |
| Gellan Gum High Acyl | 1.98 | (1.5-2) | 0.47 | (0.45-0.5) | Texturizing ingredient: |
| Gellan Gum Low Acyl | 1.6 | (1.5-2) | 0.38 | (0.35-0.4) | hydrocolloid |
| Tetrasodium | 2.3 | (2-2.5) | 0.55 | (0.5-1.0) | Buffer |
| Pyrophosphate | | | | | |
| Natural Preservative | 4.6 | (4.5-5) | I.I | (0.7-1.3) | Shelf-life enhancer |
| Isomalto- | 2.73 | (2.5-3) | 0.65 | (0.6-0.75) | Texturizing agent: |
| oligosaccharide | | | | | polysaccharide |
| Vitamin Blend | 2.18 | (2-2.3) | 0.53 | (0.5-0.6) | Fortification |
| Sunflower Lecithin | 1.68 | (1.5-1.7) | 0.38 | (0.35-0.4) | Emulsifier |
| Potassium Citrate | 0.55 | (0.4-0.6) | 0.13 | (0.1-0.15) | Buffer/cation salt |
| Kosher Salt | 0.67 | (0.5-0.8) | 0.16 | (0.1-0.2) | Texturizing agent: |
| | | | | | cation salt/flavour |
| | | | | | enhancer |
| Transglutaminase TI | 0.42 | (0.4-0.5) | 0.10 | (0.1-0.15) | Texturizing agent |
| Enzyme | | | | | |
| Magnesium Lactate | 0.42 | (0.4-0.5) | 0.10 | (0.1-0.15) | Texturizing agent: |
| | | | | | cation salt/fortification |
| Calcium Carbonate | 0.25 | (0.2-0.3) | 0.06 | (0.05-0.08) | Texturizing agent: |
| | | | | | cation salt/fortification |
| Beta Carotene | 0.08 | (0.07-0.1) | 0.02 | (0.017-0.025) | Colorant |
| Dehydrated Onion | 0.34 | (0.3-0.4) | 0.08 | (0.05-0.1) | Flavour enhancer |

Mixotrophic-borne (vegetarian-) egg analog -
A second receipt (adopted from WO2023/279189)

| | | Total | |
|---|---|---|---|
| Liquid Egg Analog | Dry Weight | Weight | |
| Ingredient | (%) | (%) | Description |
| Water | | 67.28 | Dispersant |
| A first mixotrophic- | 28.56 | 5.50 | Functional protein |
| borne protein | | | |
| Canola Oil | 37.38 | 7.20 | Dispersant |
| A first and/or a second | 18.38 | 3.54 | Functional protein |
| mixotrophic-borne | | | |
| protein | | | |
| Gellan Gum High Acyl | 1.82 | 0.35 | Texturizing ingredient: |
| Gellan Gum Low Acyl | 1.51 | 0.35 | hydrocolloid |
| Tetrasodium | 1.61 | 0.31 | Buffer |
| Pyrophosphate | | | |
| Isomaltooligosaccharide | 4.31 | 0.83 | Texturizing agent: |
| | | | polysaccharide |
| Vitamin Blend | 0.52 | 0.10 | Fortification |
| Sunflower Lecithin | 2.08 | 0.40 | Emulsifier |
| Potassium Citrate | 1.09 | 0.21 | Buffer/cation salt |
| Kosher Salt | 0.78 | 0.15 | Texturizing agent: |
| | | | cation salt/flavour |
| | | | enhancer |
| Transglutaminase TI | 0.47 | 0.09 | Texturizing agent |
| Enzyme | | | |
| Magnesium Lactate | 0.62 | 0.12 | Texturizing agent: |
| | | | cation salt/fortification |
| Calcium Lactate | 0.52 | 0.10 | Texturizing agent: |
| | | | cation salt/fortification |
| Natural Colour | 0.16 | 0.03 | Colorant |
| Dehydrated Onion | 0.21 | 0.04 | Flavour enhancer |

### Example 7

### Mixotrophic-Borne (Vegetarian-) Mayonnaise

Many popular food ingredients and products are mixtures in which particles of one substance (a “dispersed phase”) are dispersed throughout a volume of a different substance (a “dispersion medium” or “dispersion phase”); mixtures of this type are referred to herein as “colloidal” or “colloids.” The hereto provided section serve as an example provided in a non-limiting manner for any colloidal edible composition, such as blancmange, bread, butter, cake, custard, egg white foam, ice cream, jam, jelly, margarine, mayonnaise, meringue, milk, salad dressing, mayonnaise, commercial mayonnaise substitutes, alfredo sauce, and hollandaise sauce. The sauce or dressing may not contain egg, egg white, or any protein extracted from egg, and whipped cream, see WO2022165306A1.

As used herein, unless otherwise specified, the term “analog” or “analog food product” refers to a food product comprising edible fungi that bears an aesthetic, culinary, nutritional, and/or sensory equivalence or resemblance to an identified non-fungal food product. By way of non-limiting example, an “ice cream food analog product,” as that term is used herein, refers to a food product comprising edible fungi that bears an aesthetic, culinary, nutritional, and/or sensory equivalence or resemblance to conventional ice cream made from animal milk, and a “mayonnaise food analog product,” as that term is used herein, refers to a food product comprising edible fungi that bears an aesthetic, culinary, nutritional, and/or sensory equivalence or resemblance to conventional mayonnaise made using animal products.

As used along this patent, unless otherwise specified, the term “colloid” refers to a mixture in which particles of one substance (the “dispersed phase”) are dispersed throughout a volume of a different substance (the “dispersion medium”); for example, the dispersed phase can comprise or consist of microscopic bubbles, particles, etc. Where the dispersed phase and the dispersion medium of a colloid are specifically identified herein, they are separated by a hyphen, with the dispersed phase identified first, e.g., a reference herein to an “oil-water colloid” refers to a colloid in which an oil is the dispersed phase and water is the dispersion medium.

As used along this patent, unless otherwise specified, the term “emulsion” refers to a colloid in which both the dispersed phase and the dispersion medium are liquids. Examples of emulsions as that term is used herein include but are not limited to butter (when melted), margarine (when melted), mayonnaise and milk.

Mixotrophic-borne protein compositions disclosed herein are adopted from WO2021034980A1 and include sauces and dressings, such as an mixotrophic-borne eggless mayonnaise, commercial mayonnaise substitutes, gravy, sandwich spread, salad dressing or food sauce. Inclusion of mixotrophic-borne protein compositions in a sauce or dressing, and the like, can provide one or more characteristics such as binding, emulsifying, odor neutrality, and mouthfeel. In some embodiments mixotrophic-borne protein compositions is present in such sauces and dressing in an amount between 0.1% and 3% or between about 3% and about 5% w/w/or w/v. In some cases, the amount of mixotrophic-borne protein compositions in a sauce or dressing may be substantially similar to the amount of whole egg, egg-white or mixotrophic-borne protein compositions used in a commercially available or commonly used recipe. Exemplary sauces and dressing include mayonnaise, commercial mayonnaise substitutes, alfredo sauce, and hollandaise sauce. In some embodiments, the mixotrophic-borne protein compositions-containing sauce or dressing does not contain whole egg, egg white, or any other protein extracted from egg. In some cases, the sauce, dressing or other emulsified product made with mixotrophic-borne protein compositions includes at least one fat or oil and water. Exemplary fats and oils for such compositions include corn oil, safflower oil, nut oils, and avocado oil.

Mixotrophic-borne mayonnaise food analog product

| INGREDIENTS | % BY WEIGHT |
|---|---|
| Canola oil | about 58.5 |
| Water | 22 |
| White sugar | 8 |
| Salt | 3.2 |
| Vinegar | 10 |
| Mixotrophic-borne Protein | 10 |
| Mustard | 0.8 |
| total | 100 |

### Example 8

### Mixotrophic-Borne (Vegetarian-) Milk Analogs and Products Thereof

In this invention, dairy products or milk products, also known as lacticinia, are food products (whether Kosher as milk or pareve foods) made from (or containing) analogs of animal milk, such as (examples only), flowing milk, cream, butter, fermented food products, yogurt, cheese, custard food products and frozen food products, see also currently available link https://en.wikipedia.org/wiki/List_of_dairy products, incorporated herein as a reference.

The mixotrophic-borne milk analog (e.g., reconstituted product) can be a suspension, a colloid, an emulsion, a homogeneous mixture, a gel, a solid, and/or have any other suitable structure or form factor. In an example, the milk analog can be produced by emulsifying the mixotrophic-borne protein component (e.g., protein isolate and/or protein assemblies) with a lipid component. In another example, the milk analog can be produced by emulsifying the mixotrophic-borne protein component (e.g., mixotrophic-borne protein isolates and/or mixotrophic-borne protein assemblies) and a lipid component within an aqueous continuous phase. However, the milk analog can be otherwise produced. Ingredients of the mixotrophic-borne milk are selected such that approximately 4-6% contains carbohydrates, 2-4% contains fat, and 3-4% contains mixotrophic-borne protein (e.g., by weight). Otherwise, ingredients are selected such that the mixotrophic-borne milk analog formed from the reconstituted product is a milk analog for human milk (e.g., containing approximately 0.5 to 1.5 g/dL for total mixotrophic-borne protein, 1.5 to 10 g/dL for fat, and 5 to 8 g/dL for carbohydrates, see US20230000106A1).

Some of the mixotrophic-borne milk analog products are beverages. The hereto disclosed mixotrophic-borne beverage is selected from the group consisting of a ready-to-drink beverage, a mixotrophic-borne milk analog milk analog beverage, a weight management beverage, a mixotrophic-borne milk analog protein shake, and a meal replacement mixotrophic-borne milk analog drink.

In some embodiments, the beverage is cold-pressed juice.

In some embodiments, the edible material is selected from the group consisting of mixotrophic-borne milk analog skim milk, whole mixotrophic-borne milk, cream, dried mixotrophic-borne milk powder, non-fat dry mixotrophic-borne milk powder, caseinate, mixotrophic-borne protein concentrate, mixotrophic-borne protein isolate, mixotrophic-borne chocolate flavored product, mixotrophic-borne cocoa containing powder, mixotrophic-borne coffee containing product, and combinations thereof.

In some embodiments, the mixotrophic-borne milked food or mixotrophic-borne milked beverage product further comprises an ingredient selected from the group consisting of a sweetening agent, an emulsifying agent, a thickening agent, a stabilizer, a lipid material, a preservative, an antioxidant, a flavoring agent, a coloring agent, a vitamin, a mineral, and combinations thereof.

In some embodiments, the mixotrophic-borne milked food or mixotrophic-borne milked beverage product is selected from the group consisting of a mixotrophic-borne milk food bar, a nutritional supplement, a cereal-based product, a mixotrophic-borne meat analog product, mixotrophic-borne deli-meat, and a mixotrophic-borne dairy or analog product.

In some embodiments, the mixotrophic-borne milked food or mixotrophic-borne milked beverage product is yogurt, ice cream, milkshake or the like.

### Example 79

### Mixotrophic-Borne (Vegetarian-) Cheese

Cheese is a milk-containing food product. The term cheese refers hereinafter to one and all of the following: Fresh and whey cheeses, such as Corsican brocciu, Italian ricotta, Romanian urda, Greek mizithra, Croatian skuta, Cypriot anari cheese, Himalayan chhurpi and Norwegian Brunost, cottage cheese, cream cheese, curd cheese, farmer cheese, ca, chhena, fromage blanc, queso fresco, paneer, fresh goat's milk chèvre, Breingen-Tortoille, Irish Mellieriem Rochers and Belgian Mellieriem Rochers. Such cheeses are often soft and spreadable, with a mild flavour. Stretched curd cheeses such as mozzarella and halloumi, Parmesan and Grana Padano. Cooked pressed cheeses such as Swiss Emmental, Gruyère and Appenzeller, as well as the French Beaufort and Comté. Cheese that characterized by various moisture: soft to hard: soft cheese such as Brie and Neufchâtel, Semi-soft cheese such as Monastery cheeses, Havarti, Munster, Port Salut and Butterkäse., Medium-hard cheese, such as Emmental and Gruyère Gouda, Edam, Jarlsberg, Cantal, and Kashkaval/Caşcaval, and Semi-hard cheese, such as Cheddar, Edam and Gouda Colby and Monterey Jack, hard cheese, such as Grana Padano, Parmesan or Pecorino. The term also refers to molded cheese, such as soft-ripened cheeses, Smear-ripened cheese, washed-rind cheeses and blue cheeses. The term also refers to processed cheese and yellow cheese.

Cheese analog embodying this invention is a smooth, homogenous plastic mass wherein the principal mixotrophic-borne protein sources for the cheese analog is a combination of mixotrophic-borne protein (having a fat content of less than 2%) and previously dry but now hydrated edible rennet casein. The coagulated mixotrophic-borne product is present at about 15% to about 35%, preferably about 15% to about 20%, of the total cheese analog. The rennet casein is present at about 15% to about 35%, preferably at about 20% to about 30%, of the total cheese analog.

Skimmed milk, or skim milk is made when all the milkfat is removed from whole milk. It tends to contain around 0.1% fat. Those products are formed using a combination of a coagulated skim milk product (such as baker's cheese or cottage cheese) having substantially no fat (less than 2%) and rennet casein, together with water, emulsifying salts and other conventional ingredients used in cheese analog production. The cheese analog embodying this invention is a smooth, homogenous plastic mass wherein the principal protein sources for the cheese analog is a combination of coagulated skim milk (having a fat content of less than 2%) and previously dry but now hydrated edible rennet casein. Ingredients used in cheese analog production are as follows:

Mixotrophic-borne Mozzarella cheese analog

| INGREDIENTS | % BY WEIGHT |
|---|---|
| Water | 65.6 |
| Coconut oil | 24 |
| mixotrophic-borne protein | 3.34 |
| Trisodium citrate | 0.16 |
| Perfectsol 500 | 6 |
| Salt | 6 |
| Lemon salt | 0.07 |
| Aroma cream extract 7122 | 0.1 |
| Sweet cream extract 0722 | 0.12 |
| Protein percentage | 1 |

Mixotrophic-borne Mozzarella cheese
analog (adopted from CA2092890)

| INGREDIENTS | % BY WEIGHT |
|---|---|
| Water | 46.80 |
| Dry particulate rennet casein | 25.30 |
| and/or mixotrophic-borne protein | |
| skimmed milk (0.2% Fat) | 18.00 |
| Modified corn starch | 4.40 |
| Kasal (sodium aluminum phosphate) | 1.80 |
| Tricalcium phosphate | 1.40 |
| Lactic acid | 0.60 |
| Disodium phosphate | 0.50 |
| Glycerin | 0.32 |
| Adipic acid | 0.39 |
| Sorbic acid | 0.39 |
| Salt | 0.32 |
| Flavors and Color | 0.78 |
| TOTAL | 100.00 |

Mixotrophic-borne American Cheese
Analog (adopted from CA2092890)

| INGREDIENTS | % BY WEIGHT |
|---|---|
| water | 39.45 |
| rennet casein and/or mixotrophic-borne | 18.98 |
| protein | |
| skimmed milk | 17.98 |
| modified corn starch | 13.15 |
| kasal (sodium aluminum phosphate) | 1.80 |
| tricalcium phosphate | 1.25 |
| lactic acid (88\) | 1.00 |
| disodium phosphate (anhyd.) | 0.80 |
| maltodextrin | 3.00 |
| adipic acid | 0.40 |
| sorbic acid | 0.30 |
| salt | 0.75 |
| guar gum | 0.50 |
| flavors and color | 0.74 |
| TOTAL | 100.00 |

### Example 810

### Mixotrophic-Borne (Vegetarian-) Ice Cream

Ice cream milk-containing food product, namely, a sweetened frozen food typically eaten as a snack or dessert. It may be made from milk or cream and is flavoured with a sweetener, either sugar or an alternative, and a spice, such as cocoa or vanilla, or with fruit such as strawberries or peaches. It can also be made by whisking a flavored cream base and liquid nitrogen together. Food coloring is sometimes added, in addition to stabilizers.

In one embodiment of the invention, the mixotrophic-borne (vegetarian-) ice-cream comprises 2% milk analog as defined above with 3-7% mixotrophic-borne protein or lecithin, (520g-685.5g), canola oil (64.5g), cane sugar (0-142g) for a total of 750g.

### Example 911

### Mixotrophic-Borne Pasta

### Fresh and Dried Mixotrophic-Borne Pasta

500 g of pasta flour 00 (extra fine double milled flour)
55 g mixotrophic-borne protein
8 g of salt
80 ml of olive oil
water

### Example 1012

### Mixotrophic-Borne Bakery Products, Including Bread, Challah, Cake

Challah Bread with Mixotrophic-Borne Protein
1 kg of white flour
1 cube or 50 grams of fresh yeast
3/4 cup white sugar
50g mixotrophic-borne protein
1 3/4 cups of water
1/2 cup canola oil
1 tablespoon fine salt

Cake with mixotrophic-borne protein and Xanthan
gum, (total 100%, adopted from WO2021/034980)

| Ingredients | % w/w |
|---|---|
| Lecithin | 0.09 |
| All-purpose Flour | 22.61 |
| Granulated Sugar | 22.61 |
| Unsalted mixotrophic-borne butter | 25.63 |
| mixotrophic-borne sour cream as defined above | 5.03 |
| Coarse salt | 0.18 |
| Baking powder | 1.21 |
| vanilla extract | 0.37 |
| mixotrophic-borne protein | 3.41 |
| Water | 18.74 |
| Xanthan gum | 0.05 |
| Marigold yellow | 0.06 |

Sponge cake with mixotrophic-borne protein

| Ingredients | % w/w |
|---|---|
| Palm oil | 14.5 |
| White sugar | 21 |
| Oblate Chef | 1.21 |
| Invert sugar | 2.1 |
| Glycerin | 2.9 |
| Propylene glycol | 0.11 |
| mixotrophic-borne protein | 14.5 |
| Water | 12.1 |
| White flour | 29 |
| Corn flour | 0.72 |
| Vanilla extract | 0.15 |

### Example 1113

MIXOTROPHIC-BORNE CRACKER, NAMELY A FLAT,
DRY BAKED BISCUIT TYPICALLY MADE WITH FLOUR

| Ingredients | % w/w |
|---|---|
| Corn starch | 26.6 |
| Potato starch | 8.4 |
| mixotrophic-borne protein | 3.6 |
| Sugar | 3 |
| Salt | 1.8 |
| Black pepper | 1.9 |
| Sunflower oil | 9.1 |
| Water | 29.7 |
| various seeds | 4.6 |
| Flax seeds | 3 |
| Sesame | 1.9 |
| Brush seeds | 1.5 |
| Brush oil | 0.045 |

### Example 1214

### Honey Analog

Honey is a semi liquid product (water: approx.15-18%) which contains a complex mixture of carbohydrates, mainly glucose and fructose; other sugars are present as traces, depending on the floral origin. Moreover, organic acid, Lactones, amino-acids, minerals, vitamins, enzymes, pollen, wax and pigments are present. Honey is produced either from many flowers or from single flower pollens. The single flower origin should assure a better quality of the product, when it guarantees a specific and well-defined flavour and aroma. The term “honey” refers to any type of honey analog produced by any type of bee from any type of floral source, or any other source, in any region of the world. In one embodiment, the honey can include polyfloral honey analog, monofloral honey analog, or honeydew honey analog.

Honey has a content of 80-85% carbohydrates, 15-17% water, 0.3% proteins, 0.2% ashes and minor quantities of amino-acids, phenols, pigments and vitamins. Nutritionally, simple carbohydrates make up about 82.4% of honey, being primarily fructose (38.5%) and glucose (31%), with some maltose, sucrose and other sugars. Honey also contains some 17.1% water, with protein and micronutrients making up the balance (0.5%). It has been reported that pollen proteins are 7.5-35% by weight to the total weight of pollens. Measuring total honey proteins: Comparing natural honey proteins obtained in this study, even in July-Aug. that has the least percentage of protein (0.57%) and pollen protein is 0.11%, with protein of another honey (23%) that is sold in the market as a natural honey under a well-known mark and its pollen quantity is 0.01% draws our attention to considerable quantity of pollen protein of natural honey with good quality and can be regarded as a good criterion for specifying a natural honey, see Nazarian, H, Razieh T, and Ahmad M. “Origin of honey proteins and method for its quality control.” Pakistan Journal of Botany 42.5 (2010): 3221-3228.

At 25° C. (77° F.), honey with 14% water content generally has a viscosity around 400 poise, while a honey containing 20% water has a viscosity around 20 poises. Viscosity increases very slowly with moderate cooling; a honey containing 16% water, at 70° C. (158° F.), has a viscosity around 2 poises, while at 30° C. (86° F.), the viscosity is around 70 poises. With further cooling, the increase in viscosity is more rapid, reaching 600 poises at around 14° C. (57° F.). However, while honey is viscous, it has low surface tension of 50-60 mJ/m², making its wettability similar to water, glycerin, or most other liquids.

It is hence in the scope of the invention to disclosed a mixotrophic-analog to honey, comprising the following ingredients:

Mixotrophic-borne honey analog

| | Range (% wt) | Mean (% wt) |
|---|---|---|
| Water | 14-20 | 17.2 |
| Total sugars | | 79.7 |
| Monosaccharides | | |
| fructose | 30-45 | 38.2 |
| glucose | 24-40 | 31.3 |
| Disaccharides | | |
| sucrose | 0.1-4.8 | 0.7 |
| others | 2.0-8.0 | 5.0 |
| Trisaccharides | | |
| oligosaccharides | | 3.1 |
| erlose | 0.5-6.0 | 0.8 |
| melezitose | | <0.1 |
| others | 0.5-1.0 | 0.5 |
| Minerals | 0.1-0.5 | 0.2 |
| Mixotrophic-borne protein | 0.2-0.4 | 0.3 |
| Acids | 0.2-0.8 | 0.5 |

Mixotrophic-borne honey analog

| | Range (% wt) | Mean (% wt) |
|---|---|---|
| pH value | 3.2-4.5 | 3.9 |
| Viscosity | 400-20 poises* | * at 25° C. |
| surface tension | 50-60 mJ/m² | |

### Example 1315

### Production of Mixotrophic-Borne Food Products in a Closed Photobioreactor for Growing a Microorganism Culture in an Aqueous Medium

It is in the scope of the present invention wherein the mixotrophic-borne food products are produced in a closed photobioreactor for growing a microorganism culture in an aqueous medium such the one presented in WO2021149045A1.

It is in the scope of the present invention wherein a method of producing edible mixotrophic-borne proteinic product utilizable as an ingredient, beverage or food product is provided useful by utilizing step(s) inter alia consisting of fermenting a microorganism under mixotrophic conditions in closed photobioreactor for growing a microorganism culture in an aqueous medium.

It is also in the scope of the present invention wherein a cultivation system comprising at least one closed cultivation tank comprising: optic fiber based light dispersing subsystem; double walled external wall adapted to allow cooling of the liquid within the tank; nutrient dispensing unit adapted to allow dispensing of nutrients into the liquid within the tank; and gas dispensing unit adapted to allow dispensing of gases into the liquid within the tank, a natural light concentration and funneling subsystem using optical fiber; at least one optical coupler coupling the funneling subsystem outgoing optical fibers to the cultivation tank ingoing optical fibers; at least one sensing means adapted to sense parameters within the system's components; and a control unit comprising at least one computing device adapted to receive sensed data from the sensors and manage the system's processes, as presented in WO2019064291A1. This system is operative in method comprising steps of: collecting natural light using at least one lens into an optic fiber; filtering the light to remove unwanted wavelengths; funneling the light from the fiber to cultivating tank's ingoing optic fiber using an optic coupler wherein the coupler further comprise flashing mechanism; dispersing the light inside the tank using side emitting optic fibers; controlling the temperature of a liquid within the tank using a double wall in the tank; sensing organisms growing related parameters using sensors; and tracking, controlling and dispensing nutrients and gasses using a control unit comprising a computing device.

It is also in the scope of the invention wherein a closed photobioreactor for growing a microorganism culture in an aqueous medium is utilized according to technology disclosed by IL272146. The photobioreactor comprising: a vessel comprising a vessel floor, a vessel cover positioned substantially parallel thereto, and at least one vessel wall positioned perpendicular to the vessel floor and vessel cover, defining an internal cavity for containing the microorganism culture in the aqueous medium, wherein the vessel cover comprises a plurality of openings; and a plurality of transparent pipes attached to the vessel cover configured to accommodate a plurality of light sources within lumens thereof, wherein each of the transparent pipes extends through one of the openings, wherein each transparent pipe has a first open end located out of the internal cavity of the vessel, and a second end, wherein a first portion of each transparent pipe is connected to the vessel cover, and a second portion of each transparent pipe is located within the internal cavity, wherein each of the transparent pipes is substantially sealed to one of the openings, so that the internal cavity is isolated from the surrounding environment of the vessel.

It is also in the scope of the invention wherein a sealing valve is used in a technology defined in IL277523. A tubular seal for sealing between a wall and a cover of a cylindrical container. The seal extends around a centerline and longitudinally between a proximal end and a distal end, wherein the seal comprises: a membrane juncture extending around the centerline; a first membrane extending distally from the membrane juncture; a second membrane extending radially inward from the membrane juncture; and an anchoring membrane extending radially outward from the membrane juncture; wherein when the seal is positioned between the wall and the cover and no pressure is applied there against, each of the second membrane and the anchoring membrane is configured to seal against the cover, and the first membrane is configured to seal against the wall; wherein the first membrane is made of a resilient flexible material so as to bend inward toward the centerline when flow-induced pressure gradient is applied thereto by fluid flowing in the inward direction and to spontaneously restore the sealing against the wall, when the flow-induced pressure gradient ceases; and the second membrane is made of a resilient flexible material so as to bend distally when flow-induced pressure gradient is applied thereto by fluid flowing in the distal direction and to spontaneously restore the sealing against the cover, when the flow-induced pressure gradient ceases.

It is also in the scope of the invention wherein a sealing valve is used in a technology defined in IL273241. A main tubular body extending longitudinally between a proximal end and a distal end, comprising: a proximal segment extending distally from the proximal end; and a tapering segment extending from the proximal segment and tapering radially inward in the distal direction; a membrane valve portion extending around and radially away from the main tubular body, comprising: a membrane juncture, along which the membrane valve portion is attached to the main tubular body; a membrane body, extending radially away from the membrane juncture; and a membrane lip: wherein the membrane portion is resiliently biased toward the proximal direction, and is configured to bend distally when flow-induced pressure gradient is applied thereto by fluid flowing in the distal direction; the distal end is configured to transition between a distal free state, when no tube extends there-through, and a distal sealed state, when a tube extends there-through, so as to seal against the tube while no flow-induced pressure gradient is applied thereto; and the distal end is made of a resilient flexible material, configured to expand in the radial direction away from the tube extending there-through, when fluid pressure is applied to the distal end by distally-oriented fluid flow through the main body.

In one example, the photobioreactor may comprise a vessel comprising a vessel floor, a vessel cover positioned substantially parallel thereto, and at least one vessel wall positioned perpendicular to the vessel floor and vessel cover, defining an internal cavity for containing the microorganism culture in the aqueous medium, wherein the vessel cover comprises a plurality of openings; and a plurality of transparent pipes attached to the vessel cover configured to accommodate a plurality of light sources within lumens thereof, wherein each of the transparent pipes extends through one of the openings, wherein each transparent pipe has a first open end located out of the internal cavity of the vessel, and a second end, wherein a first portion of each transparent pipe is connected to the vessel cover or the vessel floor, and a second portion of each transparent pipe is located within the internal cavity, wherein each of the transparent pipes is substantially sealed to one of the openings, so that the internal cavity is isolated from the surrounding environment of the vessel.

It is also in the scope of the present invention wherein the mixotrophic-borne food products are process for growing a microorganism culture in an aqueous medium. The process comprising (a) providing the closed photobioreactor as defined above, wherein the plurality of transparent pipes are accommodating a plurality of light sources within the lumens thereof; (b) placing an aqueous biological medium comprising at least one cell population within the internal cavity; (c) operating the plurality of light sources to irradiate light into the internal cavity through the at least some of the plurality of transparent pipes, thereby growing the at least one cell population.

As used herein, the term “bioreactor” refers to any system, device, apparatus or structure capable of supporting a biologically active environment and for growing organisms such as bacteria and/or algae under controlled conditions for production of products. As used herein, the term “photobioreactor”, as used herein, means a device or system used to support a biologically active environment for the cultivation of phototrophic microorganisms, including photo synthetic microorganisms. A photobioreactor may include translucent materials that permit penetration of light therethrough, and/or may incorporate a light source to provide photonic energy input for an aqueous culture of photo synthetic microorganisms contained therein. The term “closed photobioreactor” refers to a closed system which at least temporarily isolates culture media contained therein from the surrounding environment.

These photobioreactors of the current example are configured for growing a microorganism culture in an aqueous medium. The microorganism culture may be of any microorganism species, which requires light for its cultivation, according to some embodiments. According to some embodiments, the microorganism culture is a phototroph culture, heterotroph culture, mixotroph culture or any combination thereof.

From the foregoing description, it will be apparent that variations and modifications may be made to the invention described herein to adopt it to various usages and conditions. Such embodiments are also within the scope of the following claims. The recitation of a listing of elements in any definition of a variable herein includes definitions of that variable as any single element or combination of listed elements. The recitation of an embodiment herein includes that embodiment as any single embodiment or in combination with any other embodiments or portions thereof. All patents and publications mentioned in this specification are herein incorporated by reference to the same extent as if each independent patent and publication was specifically and individually indicated to be incorporated by reference.

1.-13. (canceled)

14. An edible mixotrophic-borne product comprising a composition of mixotrophic borne edible proteins of mixotrophic microorganisms, the proteins selected from Group A of proteins and from Group B of proteins, and wherein said edible mixotrophic-borne product is characterized by: at least a two-fold increased intensity of said mixotrophic borne edible proteins selected from Group A and at least a two-fold increased intensity of said mixotrophic borne edible proteins selected from Group B, both compared to control.

15. The edible mixotrophic-borne product of claim 14, wherein said microorganisms are cultured under uninterrupted continuous mixotrophic conditions in a photobioreactor with intensity of light, wavelengths at a range of 300 and 700 nm, between 100 to 7,000 lux/liter; flux of photons to surface of said photobioreactor is between 10 to 2500 micro-Einstein (μE, m⁻²s⁻¹), growth medium along at least last three to six days of pre-harvesting comprises glucose as an organic carbon source at a concentration of 0.1 to 40 grams per liter.

16. The edible mixotrophic-borne product of claim 14, selected form a group consisting of food and beverage product(s) comprising meat/fish analog, eggs analog, milk analog, including cheese, yogurt, bakery and pasta product, including breads and cakes, honey analog and any combination, derivative and mixture thereof.

17. The edible mixotrophic-borne proteinic product of claim 14, wherein said control comprises any one of heterotrophic borne proteins of Group A if they were produced by a same microorganism grown in heterotrophic conditions and autotrophic borne proteins of Group B if they were produced by a same microorganism grown in photoautotrophic conditions

18. The edible mixotrophic-borne product of claim 14, comprising Ribulose-1,5-bisphosphate carboxylase/oxygenase (RuBisCo) where percentage of RuBisCo in the composition is at a range of 5 to 100 percentages of total dry weight.

19. The edible mixotrophic-borne product of claim 14, wherein said protein is a member of a group consisting of a whole mixotrophic microorganism, portions of said mixotrophic microorganism, water miscible extracts from said mixotrophic microorganism or portions thereof, water immiscible extracts from said mixotrophic microorganism or portions thereof, and any combination, derivatives and mixtures thereof.

20. The edible mixotrophic-borne product of claim 14, being a member of a group consisting of: an ingredient, a beverage, and a food product.

21. A method of obtaining an edible mixotrophic-borne product, the method comprising the steps of culturing microorganisms under uninterrupted continuous mixotrophic conditions in a photobioreactor with intensity of light, wavelengths at a range of 300 and 700 nm, between 100 to 7,000 lux/liter; flux of photons to surface of said photobioreactor is between 10 to 2500 micro-Einstein (μE, m⁻²s⁻¹); and providing growth medium along at least last three to six days of pre-harvesting with glucose as an organic carbon source at a concentration of 0.1 to 40 grams per liter and optionally producing a proteinic product from said edible mixotrophic-borne product.

22. The method of claim 21, wherein said culturing comprises receiving a micro microalgae culture grown on predetermined controlled mixotrophic growth conditions to a desirable growth rate, and transferring said microalgae culture into industrial-scale photobioreactor designed for commercial production purposes, for further growing under predetermined controlled mixotrophic growth conditions and production of a proteinic product.

23. The method according to claim 21, wherein a growth medium contains an organic carbon source at a concentration of about 0.1 to about 40 grams per liter.

24. The method according to claim 21, wherein the growth medium also contains organic or inorganic nitrogen and phosphate.

25. The method according to claim 21, comprising at least one of:
i. high flux of photons is maintained throughout the entire growing process, from plate stage to industrial reactor, with light intensity between about 100 to about 7,000 lux/liter;
ii. flux of photons to surface of said photobioreactor is between about 10 to about 2,500 μE, m⁻²s⁻¹;
iii. lighting wavelengths provided are in a photosynthetic range (PAR) and can include specific wavelengths in a range between about 300 and about 700 nm;
iv. Temperature is maintained between about 10 degrees and about 40 degrees Celsius; and
v. pH of culture medium is maintained within a range of about 4 to about 10.

26. The method according to claim 25, wherein said at least one is at least two.

27. The method according to claim 21, wherein said proteinic product is a member of a group consisting of a whole mixotrophic-fermented microorganism, portions of said mixotrophic-fermented microorganism, water miscible extracts from said mixotrophic-fermented microorganism or portions thereof, water immiscible extracts from said mixotrophic-fermented microorganism or portions thereof, and any combination, derivatives and mixtures thereof.

28. The method according to claim 21, wherein said proteinic product is a member of a group consisting of meat analog, food product comprising meat analog, eggs analog, food product comprising eggs analog, milk analog, food product comprising milk analog, including cheese, yogurt, bakery and pasta product, including breads and cakes, honey analog and food product and confitures comprising honey analog, and any combination, derivative and mixture thereof.

29. The method according to claim 22, wherein said edible mixotrophic-borne proteinic product is a member of a group consisting of ingredient, beverage or food product, the method comprising steps of fermenting a microorganism under mixotrophic conditions in closed photobioreactor for growing a microorganism culture in an aqueous medium.
